(19) United States
(12) Patent Application Publication
(10) Pub. No.: US 2025/0134149 A1
(43) Pub. Date: May 1, 2025

(54) OGATAEA POLYMORPHA DERIVED COMPOSITIONS AND APPLICATIONS

(71) Applicant: MICROO FOOD INGREDIENTS, S.L., Madrid (ES)

(72) Inventors: Gaston PALADINI, Rosario (AR); Martin SALINAS, Funes (AR); Henk HOOGENKAMP, Molenhoek (NL); Vivek NARISETTY, Cranfield (GB); Esteban Guillermo CORLEY, Buenos Aires (AR)

(21) Appl. No.: 18/722,004

(22) PCT Filed: Oct. 6, 2023
(86) PCT No.: PCT/IB2023/060072

§ 371 Date: Jun. 20, 2024

Related U.S. Application Data
(60) Provisional application No. 63/378,661, filed on Oct. 6, 2022.
(60) Provisional application No. 63/512,850, filed on Jul. 10, 2023.

(51) Int. Cl. A23L 31/15; A23J 3/22; A23L 23/00; A23L 27/10; A61K 36/064; C12N 1/16

(52) U.S. Cl. CPC A23L 31/15; A23J 3/227; A23L 23/00; A23L 27/10; A61K 36/064; C12N 1/16; A61K 2236/19; A61K 2236/51; A61K 2236/53

(57) ABSTRACT

The disclosure provided herein is related to the use of a methylotrophic yeast and its derivatives as a food additive and source of flavoring compounds.

### CROSS REFERENCE TO RELATED APPLICATIONS

The present application claims the priority benefit of U.S. Provisional Application No. 63/378,661, filed Oct. 6, 2022, and U.S. Provisional Application No. 63/512,850, filed Jul. 10, 2023, each of which is hereby incorporated by reference in their entirety.

### REFERENCE TO SEQUENCE LISTING SUBMITTED ELECTRONICALLY

The content of the electronically submitted sequence listing (Name: 5061_003PC02_Seqlisting_ST26.xml; Size: 11,194 bytes; and Date of Creation: Oct. 6, 2023), filed with the application, is incorporated herein by reference in its entirety.

### FIELD OF THE DISCLOSURE

The present invention is related to the use of Ogataea polymorpha as food additives, flavorant or as an ingredient that could add savory, umami, irony, and/or meaty flavor to food compositions.

### BACKGROUND

The increasing demand for alternative, more sustainable, foods not including animal derivatives for their preparations, creates the challenge to provide them a suitable flavor and taste for consumer acceptance. Imparting flavor to animal-protein analogs is a challenge for the industry of alternative and extended foods.

Yeast extracts have been widely used in the food industry to adjust the desired flavor, mouthfeel, pH, color, baking properties, chew, etc. Yeast extract is usually used as a main raw material combined with reducing sugar, vitamins, and amino acids and Maillard reactions to obtain a product having a unique flavor. The yeast commonly used for this purpose is Saccharomyces cerevisiae and to a lesser extent Cyberlindnera jadiini.

The invention disclosed herein is related to extracts, hydrolyzed extracts and/or isolated molecules derived from Ogataea polymorpha as a novel and unique yeast for food applications.

The present disclosure also relates to the production of recombinant heme protein in a methylotrophic yeast, such as Ogataea polymorpha.

Surprisingly, the inventors of the present invention developed methods to efficiently obtain recombinant heme proteins in O. polymorpha without genetically manipulating the heme biosynthetic pathway (e.g., overexpression of enzymes involved in heme biosynthesis), and without addition of exogenous heme to the culture. Through the methods of the present invention, the heme binding to the produced recombinant heme protein is greater than 50%.

### SUMMARY OF THE DISCLOSURE

In some aspects, provided herein is an Ogataea polymorpha extract comprising at least about 0.03% w/w of heme. In some aspects, the Ogataea polymorpha extract comprises about 0.03% w/w of heme. In some aspects, the Ogataea polymorpha extract comprises from about 0.03% to about 3.1% w/w of heme. In some aspects, the Ogataea polymorpha extract comprises from 0.03% to 3.1% w/w of heme. In some aspects, the Ogataea polymorpha extract further comprises at least about 0.8% in weight of glutamic acid. In some aspects, the Ogataea polymorpha extract further comprises about 0.8% in weight of glutamic acid.

In some aspects, the Ogataea polymorpha extract further comprises at least about 0.007% w/w of 5′ ribonucleotides. In some aspects, said at least about 0.007% w/w of 5′ ribonucleotides is from 5′ GMP.

In some aspects, the Ogataea polymorpha extract comprises at least about 0.8% w/w of glutamic acid and at least about 0.007% w/w of 5′ ribonucleotides. In some aspects, said 5′ ribonucleotides are 5′ GMP.

In some aspects, the Ogataea polymorpha extract further comprises γ-glutamyl peptides. In some aspects, the γ-glutamyl peptides comprises glutathione.

In some aspects, the Ogataea polymorpha extract comprises at least about 0.8% w/w of glutamic acid, at least about 0.007% w/w 5′ GMP, and at least about 0.2% w/w glutathione.

In some aspects, the Ogataea polymorpha extract comprises at least about 0.03-3% w/w of heme, at least about 0.8% w/w of glutamic acid, at least about 0.007% w/w 5′ GMP, and at least about 0.2% w/w glutathione.

In some aspects, the Ogataea polymorpha extract comprises at least about 0.8% w/w of glutamic acid, at least about 0.007% w/w 5′ GMP, and at least about 0.2% w/w glutathione and comprises substantially no heme content.

In some aspects, the Ogataea polymorpha extract further comprises at least about 0.41% w/w of histidine.

In some aspects, the Ogataea polymorpha is genetically modified for producing a recombinant heme protein. In some aspects, said heme protein is produced at least about 0.1% in weight of total protein. In some aspects, the recombinant heme protein is selected from the group consisting of: hemoglobin, myoglobin, leghemoglobin, neuroglobin, cytoglobin, cytochrome P450s, cytochrome c oxidase, ligninase, catalase, peroxidases, cytochrome a, cytochrome b, and cytochrome c. In some aspects, the recombinant heme protein is an animal derived heme protein or a plant derived protein.

In some aspects, provided herein is a hydrolyzed Ogataea polymorpha extract comprising at least about 0.03% w/w of heme.

In some aspects, provided herein is a hydrolyzed Ogataea polymorpha extract comprising from about 0.03—about 3% w/w of heme.

In some aspects, the hydrolyzed Ogataea polymorpha extract further comprises at least about 0.8% w/w of glutamic acid.

In some aspects, the hydrolyzed Ogataea polymorpha extract further comprises 5′ ribonucleotides. In some aspects, the hydrolyzed Ogataea polymorpha comprises at least about 0.007% w/w of 5′ GMP.

In some aspects, the hydrolyzed Ogataea polymorpha extract further comprises at least about 0.8% w/w of glutamic acid and at least about 0.007% w/w 5′ ribonucleotides.

In some aspects, the hydrolyzed Ogataea polymorpha comprises at least about 0.007% w/w of 5′ GMP.

In some aspects, the hydrolyzed Ogataea polymorpha extract further comprises γ-glutamyl peptides. In some aspects, the γ-glutamyl peptide is glutathione. In some aspects, the Ogataea polymorpha extract comprises at least about 0.2% w/w of glutathione.

In some aspects, the hydrolyzed Ogataea polymorpha extract comprises of at least about 0.8% w/w of glutamic acid, at least about 0.007% w/w 5′ GMP, and at least about 0.2% w/w glutathione.

In some aspects, the hydrolyzed Ogataea polymorpha extract further comprises at least about 0.41% w/w of histidine.

In some aspects, the hydrolyzed Ogataea polymorpha extract comprises at least about 0.8% w/w of glutamic acid, at least about 0.007% w/w 5′ GMP, and at least about 0.2% w/w glutathione and comprises substantially no heme content.

In some aspects, the hydrolyzed Ogataea polymorpha extract is derived from a genetically modified O. polymorpha for producing a recombinant heme protein. In some aspects, said heme protein is produced in at least 0.1% in weight of total protein. In some aspects, the recombinant heme protein is selected from the group consisting of: hemoglobin, myoglobin, neuroglobin, cytoglobin, cytochrome P450s, cytochrome c oxidase, ligninase, catalase, peroxidases, cytochrome a, cytochrome b, and cytochrome c. In some aspects, the recombinant heme protein is an animal derived heme protein or a plant derived protein.

In some aspects, provided herein is a flavor precursor mixture comprising an Ogataea polymorpha biomass, proteins, amino acids, carbohydrates, and vitamins. In some aspects, the flavor precursor mixture further comprises fat, selected from vegetable fat, animal fat, microbial fat and cell cultured fat. In some aspects, the flavor precursor mixture comprises an Ogataea polymorpha biomass containing at least about 0.03% w/w of heme. In some aspects, said Ogataea polymorpha biomass is an extract. In some aspects, said Ogataea polymorpha biomass is a hydrolyzed extract. In some aspects, said Ogataea polymorpha biomass further comprises at least about 0.8% w/w of glutamic acid. In some aspects, said Ogataea polymorpha biomass further comprises 5′ ribonucleotides. In some aspects, said Ogataea polymorpha biomass further comprises at least about 0.8% w/w of glutamic acid, at least about 0.007% w/w 5′ GMP, and at least about 0.2% w/w glutathione. In some aspects, said Ogataea polymorpha biomass further comprises at least about 0.41% w/w of histidine.

In some aspects, the flavor precursor mixtures comprises a genetically modified Ogataea polymorpha for producing a recombinant heme protein. In some aspects, said heme protein is produced at least about 0.1% in weight of total protein. In some aspects, the recombinant heme protein is selected from the group consisting of: hemoglobin, myoglobin, neuroglobin, cytoglobin, cytochrome P450s, cytochrome c oxidase, ligninase, catalase, peroxidases, cytochrome a, cytochrome b, and cytochrome c. In some aspects, the recombinant heme protein is an animal derived heme protein or a plant derived protein.

In some aspects, the flavor precursor mixture comprises at least about 1% w/w Ogataea polymorpha biomass, at least about 5% w/w monosaccharides, and at least about 15% w/w amino acids.

In some aspects, provided herein is a food product comprising an Ogataea polymorpha biomass. In some aspects, the Ogataea polymorpha biomass is a lysed biomass. In some aspects, the Ogataea polymorpha lysed biomass is a partially or fully hydrolyzed biomass.

In some aspects, the food product disclosed herein comprises about 0.001% to about 80% w/w of the Ogataea polymorpha biomass. In some aspects, said Ogataea polymorpha biomass comprises at least about 0.03% w/w of heme. In some aspects, said Ogataea polymorpha biomass further comprises at least about 0.8% w/w of glutamic acid. In some aspects, said Ogataea polymorpha biomass further comprises 5′ ribonucleotides. In some aspects, said Ogataea polymorpha biomass further comprises about 0.8% w/w of glutamic acid and 5′ ribonucleotides. In some aspects, the hydrolyzed Ogataea polymorpha comprises 5′ GMP. In some aspects, the hydrolyzed Ogataea polymorpha comprises glutathione. In some aspects, said Ogataea polymorpha biomass further comprises at least about 1% w/w of histidine.

In some aspects, the food product contains no animal derived products.

In some aspects, the food product is selected from the group consisting of: sauces, marinades, condiments, dressings, brines, broths, soups, traditional protein-foods (such as tofu, tempeh, seitan), fermented vegetables and legumes, meat analogs, meat products, extended meat products, cultured meat products, and dietary supplements.

In some aspects, provided herein is a food product comprising a flavor precursor mixture comprising an Ogataea polymorpha biomass.

In some aspects, provided herein is a dietary supplement containing an O. polymorpha biomass comprising at least about 0.03% w/w of heme. In some aspects the O. polymorpha biomass comprises about 0.03% of heme. In some aspects the O. polymorpha biomass comprises from about 0.03% to about 3% w/w of heme. In some aspects the O. polymorpha biomass comprises from 0.03% to 3% w/w of heme. In some aspects, the O. polymorpha biomass is a lysed biomass. In some aspects, the O. polymorpha lysed biomass is further hydrolyzed. In some aspects, the hydrolyzed biomass is further filtrated to obtain a soluble fraction containing amino acids, peptides, polypeptides smaller than about 25, about 20, about 15, about 10, about 5, about 2, about 1, or about 0.5 kDa. In some aspects, the hydrolyzed biomass is further filtrated to obtain an insoluble fraction containing carbohydrates and/or cell membrane compounds and/or cell wall compounds smaller than about 25, about 20, about 15, about 10, about 5, about 2, about 1, or about 0.5 kDa.

In some aspects, provided herein is a method to obtain an Ogataea polymorpha extract comprising: a) culturing and propagating O. polymorpha cells; b) lysing the cells; c) adjusting the pH if needed; and d) drying and/or concentrating by removing water to obtain a solid O. polymorpha extract.

In some aspects, the method further comprises the deactivation of acidic phosphatases after the lysis of the cells.

In some aspects, the method comprises hydrolyzing endogenous proteins and nucleic acids in order to obtain molecules size with no more than about 25, about 20, about 15, about 10, about 5, about 2, about 1, or about 0.5 kDa.

In some aspects, the method comprises keeping the temperature below about 90° C., about 85° C., about 80° C., about 75° C., about 70° C., about 65° C., about 60° C., about 55° C., or about 50° C.

In some aspects, the method comprises the filtration of the hydrolyzed O. polymorpha extract to obtain a soluble and insoluble fraction. In some aspects, the soluble fraction comprises particles. In some aspects, the soluble particles include amino acids, peptides, and/or polypeptides smaller than about 100, about 50, about 25, about 20, about 15, about 10, about 5, about 2, about 1, about 0.5 kDa.

In some aspects, the insoluble fraction comprises particles. In some aspects, the insoluble particles include carbohydrates, cell membrane compounds, and/or cell wall compounds smaller than about 100, about 50, about 25, about 20, about 15, about 10, about 5, about 2, about 1, and/or about 0.5 kDa.

In some aspects, provided herein is a method for preparing a flavorant derived from O. polymorpha comprising: a) obtaining an O. polymorpha biomass; b) adding and mixing carbohydrates, fat and a source of amino acids to the O. polymorpha biomass; c) inducing a thermal treatment at about 100° C. to about 130° C. for about 30 minutes to about 180 minutes to said biomass to obtain the flavorant derived from O. polymorpha. In some aspects, said O. polymorpha biomass is previously lysed in order to obtain an extract. In some aspects, said O. polymorpha extract is further hydrolyzed. In some aspects, the hydrolyzed O. polymorpha extract is filtered to obtain a soluble and insoluble fraction, wherein the soluble fraction is used to obtain the flavorant. In some aspects, the carbohydrates are selected from the group consisting of monosaccharides, disaccharides, trisaccharides, tetrasaccharides, pentasaccharides, oligosaccharides, and polysaccharides. In some aspects, the fat is selected from the group consisting of: animal fat, insect fat, fungal fat, vegetable fat, plant fat, synthetic fat, and microbial fat. In some aspects, the source of amino acids is selected from the group consisting of: native proteins, partially hydrolyzed proteins and free amino acids.

In some aspects, the method further comprises the step of inducing the O. polymorpha cell lysis to obtain an extract. In some aspects, the method further comprises the step of inducing the proteolysis of the extract obtained. In some aspects, the method further comprises the step of inducing the hydrolysis of the nucleic acids of said extract. In some aspects, the method further comprises the step of inducing the conversion of amino acids into glutamic acid.

In some aspects, provided herein is a method of producing a heme-protein comprising greater than about 50% of heme loading. In some aspects, the method comprises the following steps: a. providing a culture of a transgenic O. polymorpha comprising a nucleic acid encoding a heme protein operatively linked to a methanol-inducible promoter; b. culturing said transgenic O. polymorpha without adding exogenous heme to the culture; and c. isolating and purifying the heme protein.

In some aspects, the transgenic O. polymorpha does not comprise an exogenous transcriptional activator of the heme biosynthetic pathway or an exogenous component of the heme biosynthetic pathway.

In some aspects, the heme protein is selected from the group consisting of animal derived heme protein and plant derived heme protein.

In some aspects, said animal derived heme protein is selected from the group consisting of bovine derived heme protein, porcine derived heme protein, ovine derived heme protein, equine derived heme protein, and caprine derived heme protein.

In some aspects, said animal derived heme protein is selected from the group consisting of: hemoglobin, myoglobin, neuroglobin, cytoglobin, cytochrome P450s, cytochrome c oxidase, ligninases, catalase, peroxidases, cytochrome a, cytochrome b, and cytochrome c.

In some aspects, said nucleic acid comprises a nucleic acid sequence comprising at least about 70% identity to the nucleic acid sequence of SEQ ID NO: 1.

In some aspects, said nucleic acid is codon-optimized, wherein the nucleic acid comprises a nucleic acid sequence comprising at least about 70% identity to the nucleic acid sequence of SEQ ID NO: 2.

In some aspects, said nucleic acid comprises a nucleic acid sequence comprising at least 70% identity to the nucleic acid sequence of SEQ ID NO: 1.

In some aspects, said nucleic acid is codon-optimized, wherein the nucleic acid comprises a nucleic acid sequence comprising at least 70% identity to the nucleic acid sequence of SEQ ID NO: 2.

In some aspects, provided herein is a method of producing an animal derived heme protein with greater than about 50% of heme loading. In some aspects, the method comprises a. introducing a nucleic acid construct into an O. polymorpha strain, wherein said nucleic acid construct comprises a promoter operatively linked to a nucleic acid encoding an animal derived heme protein; b. culturing said transgenic cells of O. polymorpha without adding exogenous heme molecules to the culture; and c. isolating the animal heme protein from the culture.

In some aspects, the transgenic O. polymorpha does not comprise an exogenous transcriptional activator of the heme biosynthetic pathway or an exogenous component of the heme biosynthetic pathway.

In some aspects, said animal derived heme protein is selected from the group consisting of: bovine derived heme protein, porcine derived heme protein, ovine derived heme protein, equine derived heme protein, and caprine derived heme protein.

In some aspects, said animal derived heme protein is selected from the group consisting of: hemoglobin, myoglobin, neuroglobin, cytoglobin, cytochrome P450s, cytochrome c oxidase, ligninases, catalase, peroxidases, cytochrome a, cytochrome b, and cytochrome c.

In some aspects, provided herein is a method of producing a recombinant bovine myoglobin comprising greater than about 50% of the recombinant bovine myoglobin loaded with heme. In some aspects, the method comprises a. introducing a nucleic acid encoding for a recombinant bovine myoglobin operatively linked to a promoter into an O. polymorpha yeast cell; b. culturing said O. polymorpha containing said recombinant bovine myoglobin nucleic acid to promote the expression of the recombinant myoglobin without adding heme to the culture, thus expressing the recombinant myoglobin.

In some aspects, the transgenic O. polymorpha do not comprise an exogenous transcriptional activator of the heme biosynthetic pathway or an exogenous component of the heme biosynthetic pathway.

In some aspects, the method further comprises extracting and purifying said recombinant bovine myoglobin.

In some aspects, provided herein is a food composition comprising any of the heme proteins disclosed herein or any of the bovine myoglobin proteins disclosed herein.

In some aspects, provided herein is a meat analog food composition comprising a lysate of any of the cultured O. polymorpha provided herein.

In some aspects, provided herein is a meat analog food composition comprising any of the heme proteins disclosed herein or any of the bovine myoglobin proteins disclosed herein.

### BRIEF DESCRIPTION OF THE DRAWINGS

FIG. 1 (FIG. 1). Optimization of media parameters and process conditions for improved heme biosynthesis upon O. polymorpha cultivation. FIG. 1A represents the yield of heme (per gram cell dry weight) obtained from O. polymorpha cultivated on different carbon sources with and without methanol induction. FIG. 1B represents the heme yields (per gram cell dry weight) obtained from O. polymorpha cultivated on different nitrogen sources with methanol induction and glycerol as the carbon source.

FIG. 1C represents the heme yields (per gram cell dry weight) obtained from O. polymorpha cultivated on optimal carbon and nitrogen sources at 150 L scale bioreactor volumes during semi-pilot trials.

FIG. 2 (FIG. 2). FIG. 2 represents the expression plasmid pFPMT121 which has been used for the generation of recombinant producer strains. pFPMT121 and its derivatives are used as circular plasmids for the transformation of O. polymorpha RB11 and ALU3 strains.

FIG. 3 (FIG. 3). FIG. 3 represents the B14 plasmid, a derivative of pFPMT121 without the antibiotic resistance gene, which is used in this work for transformation of O. polymorpha RB11 strains.

FIG. 4 (FIG. 4). FIG. 4 represents the plasmid map displaying the synthetic gene (Bovine myoglobin), an origin of replication (Col E1), restriction enzyme sites for digestion (EcoRI and BamHI), and an antibiotic resistance gene (Ampicillin AmpR).

FIG. 5 (FIG. 5). FIG. 5 represents the final transformation vector containing the promoter element (FMDp), codon optimized bovine myoglobin gene (BtMG), and transcriptional terminator (MOXt) used for transformation of O. polymorpha RB11 strains.

FIG. 6 (FIG. 6). FIG. 6 represents the strain free imaging of an SDS-PAGE gel used for the screening and selection of the positive transformants through densitometric analysis.

FIG. 7 (FIG. 7). FIG. 7 represents the amino acids sequence similarity (CLUSTAL W alignment) between the native Bovine myoglobin and Myoglobin expressed in O. polymorpha RB11 strains.

FIG. 8 (FIG. 8). FIG. 8 represents the SDS PAGE analysis of the samples collected at different intervals pre-& post-methanol induction.

FIG. 9 (FIG. 9). FIG. 9 represents the western blotting analysis of the native Bovine myoglobin and Myoglobin expressed in O. polymorpha RB11 strains. The lanes 1-3 are standard myoglobin samples with concentrations 0,0125, 0.025, and 0.05%, and lanes 5-8 are different dilutions (400×, 200×, 100× and 50×) of recombinant myoglobin samples purified and concentrated through ultrafiltration.

FIG. 10 (FIG. 10). FIG. 10 is a photograph of the O. polymorpha yeast extract.

### DETAILED DESCRIPTION

The terminology used herein is for the purpose of describing particular aspects only and is not intended to be limiting.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

Throughout this disclosure, various aspects of this disclosure are presented in a range format. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

As used herein, the term “O. polymorpha biomass” is related to the accumulated yeast or accumulated yeast cells, with or without the media culture used for the cultivation and propagation of the yeast.

The term “lysate” or “extract” refers to a media containing a mixture and/or a solution of cell contents, cell walls remnants and any culture media component attached to the cell exterior via specific binding, which was not removed before the lysis process that resulted from any method of cell lysis.

The term “lysis” refers to the rupture of the plasmatic membrane and if present, the cell wall of a cell such that a significant amount of intracellular material can escape to the extracellular space. Lysis can be performed using electrochemical, mechanical, osmotic, thermal, enzymatic, chemical, electrical, radiation, viral, or microbial means. In some aspects, the methods described herein comprise performing a lysis of cells or microorganisms as described herein in order to separate a chemical or mixture of chemicals from the contents of a bioreactor.

The invention disclosed herein is related to the use of the yeast O. polymorpha and its derived compounds as flavorant and the application in food products. The yeast O. polymorpha was previously referred to as Hansenula polymorpha, Hansenula angusta, Pichia angusta, Candida thermophila, Ogataea thermophila and Torulopsis methanothermo.

As used herein, O. polymorpha derived compounds is related to O. polymorpha cells, cell parts, cell fractions, cell components (proteins, amino acids, vitamins, carbohydrates, minerals, lipids) and the use of its to obtain flavorant precursors, flavorants and food products.

As used herein, a flavoring (or flavouring), also known as flavor (or flavour) or flavorant, is a food additive used to improve the taste, mouthfeel or smell of food.

The term “yeast extract” refers to the product obtained from the lysis of yeast. In the invention disclosed herein the yeast extract is derived from Ogataea polymorpha, therefore can be referred to as “Ogataea polymorpha extract”. In some aspects, the term “Ogataea polymorpha extract” refers to a soluble and/or insoluble Ogataea polymorpha extract. In some aspects, the term “Ogataea polymorpha extract” refers to a soluble and/or insoluble Ogataea polymorpha extract where a transgenic protein is still present. In some aspects, the term “Ogataea polymorpha extract” refers to the soluble or insoluble remnants of the lysed Ogataea Polymorpha biomass from where a transgenic protein, such as myoglobin, has been fully or partially removed. In some aspects, the Ogataea polymorpha extract can be used as a flavorant precursor, or flavoring agent. In some aspects, the Ogataea polymorpha extract can be used as a dietary supplement.

In some aspects, the lysis mediated by enzymes to cause the disruption of the cell membrane may also cause a partial hydrolysis of some components of the cell, such as proteins. In those cases, the term “Ogataea polymorpha extract” refers also to a partially or fully hydrolyzed Ogataea polymorpha biomass.

The term “hydrolyzed yeast extract” refers to a yeast extract further subjected to the hydrolysis of its contents, preferably to the hydrolysis of its macromolecules such as the nucleic acids, and proteins. In some aspects, the polymers contained in the Ogataea polymorpha cells are partially hydrolyzed. A partial hydrolysis refers to the hydrolysis of about 5%, 10%, 15%, 20%, 25%, 30%, 35%, 40%, 45%, 50%, 55%, 60%, 65%, 70%, 75%, 80%, 85%, 90%, 95%, or 99%. In some aspects, “hydrolyzed yeast extract” refers to a soluble and/or insoluble hydrolysed remnant of Ogataea polymorpha extract. In some aspects, “hydrolyzed yeast extract” refers to soluble and/or insoluble hydrolysed remnants of Ogataea polymorpha extract where a transgenic protein was present or is still present. In some aspects, “hydrolyzed yeast extract” refers to the hydrolysed remnants of the lysed yeast biomass from where a transgenic protein, such as myoglobin, has been removed before hydrolysis. In some aspects, the hydrolyzed Ogataea polymorpha extract can be used as a flavorant precursor, or flavoring agent.

The term “glutamic derived compounds” refers to molecules containing the amino acid glutamic acid (e.g., glutamyl peptides, glutathione), its salts and molecules obtained from the reaction of the glutamic acid.

The term “glutamyl peptides” refers to peptides containing two or more amino acid residues, wherein at least one of the amino acid residues is glutamic acid, γ-glutamic acid or it salts.

The terms “flavor composition” and “flavor precursors mixture” in the present disclosure, refers to a mixture of compounds capable of imparting a desirable flavor, aroma, sensation, and/or mouthfeel.

The term “artificial meat” or “meat substitute” or “imitation meat” or “meat analogue” or “meat-like food product” as used herein refers to a food product that is not derived from an animal, or that contains a substantial amount of non-animal protein source, but has structure, texture, aesthetic qualities, and/or other properties comparable or similar to those of animal meat, including livestock (e.g., beef, pork), game (e.g., venison), poultry (e.g., chicken, turkey, duck), and/or fish or seafood substitutes/analogues. The term refers to uncooked, cooking, and cooked meat-like food products. These terms also encompass cultured meat products.

As used herein, the term “non animal product” or “free of animal product” means the absence of any material directly derived from an animal, e.g., an organ, tissue, cells, proteins, nucleic acids. This definition doesn't apply to animal products produced in non-animal hosts (e.g., genetic and/or amino acid sequences derived from the sequencing of animal genetic material or the corresponding proteins).

As used herein, the term “holoprotein” refers to a conjugated functional protein bound to a ligand or a prosthetic group.

As used herein, the term “apoprotein” refers to the non-functional protein not bound to a ligand or a prosthetic group.

The term “heme proteins” include proteins that have the ability of binding a heme prosthetic group to their structure. As used herein, the term “heme protein” also refers to critical components of flesh of an animal and/or animal proteins, and can provide color and taste to plant-based meat products. Myoglobin and hemoglobin, considered heme proteins, are oxygen-binding proteins in animals. Also, as used herein, the term heme protein refers to heme containing proteins, wherein the term containing means the heme is linked through covalent or non-covalent bonds to the protein. As used herein, the term heme protein refers to not only the full-length protein but also fragments or variants thereof.

As used herein, the term “producing” refers to the ability of a yeast (e.g., Ogataea polymorpha) to express a protein of interest. In some aspects, the protein of interest is transgenic. In some aspects, the protein of interest is isolated from the yeast.

As used herein, the term “culturing” or “propagating” refers to the growth of yeast (e.g., Ogataea polymorpha) under suitable conditions for the production of the desired end-product(s) (e.g., an animal heme protein).

As used herein, “derived from” refers to the source of the flavorants, flavorant precursor, or flavoring agent. For example, a flavorant that is “derived from an Ogataea polymorpha” refers to flavorants that include one or more portions of an Ogataea polymorpha (e.g., an animal heme protein isolated from said Ogataea polymorpha).

As used herein the term “animal heme protein” or “animal-derived heme protein” refers to heme proteins from animals such as bovine, porcine, ovine, equine, and caprine. The term “animal-derived heme proteins” or “animal heme proteins” excludes the human-derived heme proteins. According to some aspects of the present disclosure, the animal heme proteins comprise and/or are selected from the group consisting of: heme proteins involved in the oxygen transport, such as hemoglobin, myoglobin, neuroglobin, and cytoglobin; enzymes having a prosthetic heme group such as cytochrome P450s, cytochrome c oxidase, ligninases, catalases, and peroxidases, as well as heme proteins involved in the electron transport chain, such as cytochrome a, cytochrome b, and cytochrome c.

As used herein, the term “plant derived heme protein”, means heme proteins from monocot or dicots plants such as Nicotiana tabacum or Nicotiana sylvestris (tobacco); Zea mays (corn), Arabidopsis thaliana, a legume such as Glycine max (soybean), Cicer arietinum (garbanzo or chickpea), Pisum sativum (pea), Phaseolus vulgaris (common bean) Vigna unguiculata (cowpea), Vigna radiata (mung beans), Lupinus albus (lupin), or Medicago sativa (alfalfa); Brassica napus (canola); Triticum sps. (wheat, including wheat berries, and spelt); Gossypium hirsutum (cotton); Oryza sativa (rice); Zizania sps. (wild rice); Helianthus annuus (sunflower); Beta vulgaris (sugarbeet); Pennisetum glaucum (pearl millet); Chenopodium sp. (quinoa); Sesamum sp. (sesame); Linum usitatissimum (flax); Lactuca sativa (Lettuce); Spinacia oleracea (Spinach); or Hordeum vulgare (barley).

As used herein, the term “recombinant heme protein” is a protein obtained from a transgenic organism, where the recombinant protein is encoded by exogenous cDNA encoding the heme protein. As used herein, the term “exogenous nucleic acid” is used herein interchangeably with “recombinant nucleic acid” and/or “heterologous gene”.

The term “transgenic”, “recombinant”, “genetic engineered”, “genetic modified”, “modified” related to a yeast (e.g., O. polymorpha) means the yeast has been transformed or transduced with one or more nucleic acids (recombinant sequences). The term “transformation” refers to a process by which a recombinant sequence is introduced and expressed in a yeast cell by using non-viral vectors.

Also, as used herein, the term “heme loading” refers to the amount of heme bound to the apo-heme recombinant protein. For example, Zhang et al. expressed porcine myoglobin in Pichia pastoris and supplemented with 150 mg/L of exogenous heme in the cultivation medium and obtained 0.22 mol of heme per mol of myoglobin, which represents 22% heme-loading. (Zhang, B., et al., Efficient secretory expression and purification of food-grade porcine myoglobin in Komagataella phaffii, Journal of Agricultural and Food Chemistry, 69 (35), 10235-10245 (2021)). Theoretically, one mol of myoglobin could bind one mol of heme which represents 100% heme-loading. The amount of heme-bound heme-protein can be determined by any of the known methods, for example those described by Hopp, M. T., et al., Heme Determination and Quantification Methods and their Suitability for Practical Applications and Everyday Use. Anal. Chem. 92, 14, 9429-9440 (2020).

According to an aspect of the present disclosure, the heme moieties present in the cell, culture media, lysate, hydrolyzed lysate and filtered hydrolyzed lysate may be free or bound to amino acids, peptides, oligopeptides, polypeptides, nucleotides, oligonucleotides, nucleic acids, carbohydrates like monosaccharides, disaccharides, oligosaccharides, polysaccharides, and/or trapped in a fat structure.

Ogataea polymorpha.

Methods for transformation of O. polymorpha are well known in the art, and include chemical transformation, electroporation, transduction, and biolistic particle delivery. For example, the plasmids applied for chemical transformation are incorporated or integrated into the yeast genome by homologous recombination (Gellissen, et al., New yeast expression platforms based on methylotrophic Ogataea polymorpha and Pichia pastoris and on dimorphic Arxula adeninivorans and Yarrowia lipolytica-a comparison, FEMS Yeast Res. 5 (11): 1079-96 (November 2005); Sohn, et al., A family of telomere-associated autonomously replicating sequences and their functions in targeted recombination in Hansenula polymorpha DL-1, J. Bacteriol. 181:1005-1013 (1999)).

O. polymorpha has many alternative names in the art, such as Candida thermophila, Hansenula angusta, Hansenula polymorpha, Ogataea thermophile, and Torulopsis methanothermo. (See speciesfungorum.org/Names/SynSpecies.asp?RecordID=362660, citing K. S. Shin, Y. K. Shin, J. H. Yoon & Y. H. Park, Int. J. Syst. Evol. Microbiol. 51 (6): 2168 (2001); Wick., Tech. Bull. U.S. Dep. Agric. 1029:31 (1951); Morais & M. H. Maia, An. Esc. Sup. Quim. Univ. Recife 1:16 (1959); G. Peter, Tornai-Leh., K. S. Shin & Dlauchy, FEMS Yeast Res. 7 (3): 495 (2007); and Urakami, Trav. Sous-Sect. Troïtzk.-Khiakta, Sect. Pays d'Amour Soc. Imp. Russe Géogr.: 1 (1975)).

O. polymorpha, is considered as a protein factory, a ubiquitous, non-conventional methylotrophic yeast, well-known as a model yeast strain for peroxisome biology. However, its unusual gene regulations associated with abiotic stress tolerance, oxidating circumstances, methanol metabolism, heavy metal resistance and nitrate assimilation define the strain as a potent candidate for further understanding its role in food and feed applications. The methylotrophic yeasts can grow in an extreme environment. O. polymorpha can tolerate temperatures of 45° C. and higher, and the strain capability of growth on methanol as a sole energy and carbon source is enabled by its methanol utilization pathway that is shared by all known methylotrophic yeasts. Gene expression is subject to a carbon source dependent repression/de-repression/induction mechanism conferred by properties of methylotrophic yeast promoters. There are specific promoters that are repressed by glucose, de-repressed by glycerol, and induced by methanol.

The cultivation of methylotrophic yeasts is known in the art (Ubiyvovk et al., Optimization of glutathione production in batch and fed-batch cultures by the wild-type and recombinant strains of the methylotrophic yeast O. polymorpha DL-1, BMC Biotechnol. 11, 8 (2011); Scheidle et al., High-throughput screening of O. polymorpha clones in the batch compared with the controlled-release fed-batch mode on a small scale, FEMS Yeast Research, 10 (1), 83-92 (2009)). In some aspects, the carbon source utilized for the production of recombinant myoglobin is glycerol. But as the promoter applied is methanol regulated, under these circumstances it may be desirable to supplement methanol to the culture media. The methanol requirement can be replaced through the substitution of methanol-inducible promoter with constitutive or sugar-inducible specific promoters. Thus, O. polymorpha can be cultivated by supplementing various substrates like glucose, glycerol, xylose, and cellobiose as the sole carbon sources.

The term “cultivation” herein designates the growing of recombinant O. polymorpha strains under optimum physical, chemical, and operational parameters for the high cell growth and protein production.

In some aspects, the O. polymorpha yeast cells can be cultivated under different physical conditions where the temperature of the surrounding medium can be maintained from about 25° C. to about−50° C. and the pH of the medium can be maintained from about 2.5 to about 7.5 using acid or alkali solutions.

In some aspects, the O. polymorpha cells can be grown in the presence of one or more of the following carbon sources: methanol, glycerol, glucose, galactose, fructose, sucrose, xylose, arabinose, or the crude glycerol, post-harvest agricultural residues (lignocellulosic) hydrolysates, post fermentation fiber and/or protein rich-residue and its hydrolysates, starchy hydrolysates, duckweed, seaweed or algal biomass hydrolysates and other renewable feedstocks.

In some aspects, the O. polymorpha cells can be grown by supplementing with one or more of the following nitrogen sources: ammonium phosphate, ammonium sulfate, animal, plant, and fungal hydrolysates like meat extract, milk protein hydrolysates, com protein hydrolysates, soy protein hydrolysates, pea protein hydrolysates, rice protein hydrolysates, corn peptone, soy peptone, potato protein hydrolysates, yeast hydrolysates, bacterial hydrolysates, fungal hydrolysates and extracts of malted grains.

In some aspects, the growth of O. polymorpha yeast biomass is supplemented with macro and micronutrients, vitamins, and minerals when grown on synthetic or chemically defined medium.

In some aspects, the optimal carbon and nitrogen sources for improved yeast biomass growth and heme production is glycerol and corn protein hydrolysate powder.

The culture medium optimized for the cultivation and expression of a recombinant heme-protein in a genetically modified O. polymorpha can comprise, for example:
(1) Pre-inoculum (YPD) medium: 2% soy peptone; 1% yeast extract; 2% glucose; 2% agar for plates; 100 mg/l adenine.
(2) Preparation instructions: In accordance with the media preparation protocol, each substance is weighed separately. The individual components are put in a mixing container, and water is added until it reaches the weight noted in the media preparation protocol. When all the components have been fully dissolved, pH and conductivity are measured and recorded in the media preparation protocol. Further the suspension is sterilized in an autoclave (20 min. 121° C.).
(3) Fermentation or production medium (SYN6):-13.32 g/L Ammonium dihydrogen phosphate; 3 g/L Magnesium sulphate; 3.32 g/L Potassium chloride; 0.33 g/L Sodium chloride; 20 g/L Glycerol; 20 mL (100× Calcium chloride); 20 mL Micro elements 100× solution (6.65 g/L EDTA; 6.65 g/L ammonium iron sulphate; 0.55 g/L copper sulphate; 2 g/L zinc sulphate; 2.65 g/L manganese sulphate); 20 mL Vitamin 100× solution (0.04 g/L D-biotin; 13.35 g/L thiamine hydrochloride); 20 mL Trace element 100× solution (65 mg/L, nickel sulphate; 65 g/L cobalt chloride; 65 g/L boric acid; 65 g/L potassium iodide; 65 g/L sodium molybdate).
(4) Preparation instructions: In accordance with the media preparation protocol, each substance is weighed separately. The individual components are put in a mixing container, and water is added until it reaches the weight noted in the media preparation protocol. When all the components have been fully dissolved, pH and conductivity are measured and recorded in the media preparation protocol.
(5) After the sterilization in autoclave, the following stock solutions are added to the reactor: 20 mL microelement solution; 20 mL vitamin solution; 20 mL calcium chloride solution; 20 mL trace element solution.
(6) Cultivation: Once the pre-inoculum is added to the production medium, the cultivation is carried out at 37° C., pH 4.8, dissolved oxygen concentration of 30% is maintained with a stirrer speed between 200-1400 rpm.
(7) 10% Structol J 673 is used as an antifoaming agent to prevent any foam deposition or formation; and pH is maintained at 4.8 by addition of 12.5% alkali (25% Ammonia solution), and 28% acid (85% phosphoric acid) solution. For the derepression, 75% w/v glycerol is fed into the reactor.

The term “parameters” as used herein designates the concentrations of chemical components like carbon, nitrogen, metal and non-metal sources, the physical conditions like pH, temperature, dissolved oxygen levels, and the operational parameters like time of incubation, stirring (rotations per minute (RPM)), aeration, etc.

In some aspects, the cultivation is initiated as batch mode with 2% w/v glycerol as the carbon source and converted into fed-batch mode with a linear feeding rate of 2-6 g/L/h, once the concentration of glycerol in the production media is less than 10 g/L. During the cultivation the substrate feed rate and the dissolved oxygen (DO) concentration in the reactor is adjusted, such a way that 20-40% DO measurement is maintained throughout the incubation time at maximum stirrer speed. The physical parameters are maintained at 37° C. and pH 5.5 throughout the cultivation.

In some aspects, the downstream processing of the yeast cell biomass harvested from the bioreactor using centrifugation, sedimentation, filtration or other mechanical means is the basis for the production of flavor, aroma, and nutritional ingredients.

In some aspects, provided herein is the use of O. polymorpha cells to create extracts, hydrolyzed extracts, and/or purified extracts to be used to further create flavorings. According to the scope of the disclosure, the biomass, extract, hydrolyzed extract, and/or specific fractions from the hydrolyzed extract, Maillard reaction precursor mixtures, Maillard reaction products, and flavorants derived from O. polymorpha are also called “O. polymorpha derived products”. In some aspects, the O. polymorpha biomass is directly used, in a wet or dried state, for the production of a flavorant, nutritional additive or a food product. In some aspects, the O. polymorpha biomass is lysed to obtain an extract, which is used to create a flavorant or a food product. In some aspects, the O. polymorpha extract is hydrolyzed for further use in flavorings or food products. In some aspects, the yeast extract or hydrolyzed yeast extract is subjected to a filtration step to concentrate a specific fraction. In some aspects the flavorant is the food product. In some aspects, the flavorant is used to confer a characteristic flavor to a food product. In some aspects, the O. polymorpha biomass is exposed to solvent extraction to concentrate and purify specific solvent soluble components like heme, nucleotides and/or amino acids.

In some aspects, provided herein is an O. polymorpha genetically modified to produce a recombinant heme protein.

In some aspects, provided herein is an O. polymorpha genetically modified to produce a recombinant heme protein, wherein the recombinant heme protein has been removed through filtration, precipitation or any other known separation method from the cell lysate. In some aspects, the remaining components are hydrolyzed to generate the hydrolyzed yeast extract and specific fractions thereof.

The term “transgenic”, “recombinant”, “genetic engineered”, “genetically modified”, “modified” related to a yeast (e.g., O. polymorpha) means the yeast has been transformed or transduced with one or more exogenous nucleic acids (recombinant sequences). The term “transformation” refers to a process by which a recombinant sequence is introduced and expressed in a yeast cell by using non-viral vectors.

The term “regulatory element” comprises a promoter which affects RNA polymerase binding. A “promoter”, as used herein, designates a DNA fragment which controls the initiation point and the initiation frequency of transcription (RNA synthesis) of a gene located under control of the promoter element in the host organism. The promoter elements that can be considered for carrying out the methods disclosed herein include, among others, the formate dehydrogenase (FMD) (SEQ ID NO: 3), methanol oxidase (MOX) (SEQ ID NO: 4), alcohol oxidase I (AOX1), glyceraldehyde-3-phoshate dehydrogenase (GAP), alcohol dehydrogenase (ADH1), translation elongation factor (TEF1), hexokinase (GLK), glucose-6-phosphate isomerase (GPI), fructose-1,6-bisphosphate aldolase (FBA), triosephosphate isomerase (TRI), phosphoglycerate mutase (PGM), pyruvate kinase (PYK), pyruvate dehydrogenase (PDH), isocitrate lyase (ICL1), L-rhamanoate dehydratase (LRA3), L-2-keto-3-deoxyrhamnonate (LRA4), glycosylphosphatidylinositol (GPI) anchored protein (GCW14), maltase (MAL), dihydroxyacetone synthase (DAS), alcohol dehydrogenase (ADH2), 6-phosphogluconate dehydrogenase (PGD), transaldolase (TAL), ribulose phosphate epimerase (RPE), catalase (CAT), superoxide dismutase (SOD), trehalose-6-phoshate (TPS1), and plasma membranse ATpase pump (PMA1) promoters from O. polymorpha, the MOX, AOX1 and GAPI promoters from Pichia pastoris and the ADHI, pyruvate decarboxylase (PDC1), GAPI and glycerol update protein (GUP1 & GUP2), from S. cerevisiae.

A “transcription terminator” as used herein, designates a DNA fragment that contains signaling structures for RNA polymerase, which result in transcription termination. Examples of usable terminator elements are the MOX (SEQ ID NO: 4), amine oxidase (AMO) or phosphate starvation (PHO1) terminator from O. polymorpha. In some aspects, the nucleic acid construct comprises a FMD promoter (SEQ ID NO: 3).

In some aspects, the nucleic acid construct comprises a nucleic acid sequence comprising at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 3.

In some aspects, the nucleic acid construct comprises a nucleic acid sequence comprising at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 3.

In some aspects, the nucleic acid construct comprises a MOX promoter (SEQ ID NO: 4).

In some aspects, the nucleic acid construct comprises a nucleic acid sequence comprising at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 4.

In some aspects, the nucleic acid construct comprises a nucleic acid sequence comprising at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4.

In some aspects, the nucleic acid construct comprises a marker. In some aspects, the marker is an auxotrophic marker or a dominant marker. In some aspects, auxotrophic markers can be: Sc LEU2*(Leucine auxotrophy), Sc URA3 (Uracil auxotrophy) (SEQ ID NO: 5), Hp URA3 (Uracil auxotrophy), Hp ADE11 (Adenine auxotrophy), Hp MET6 (Methionine auxotrophy), Hp LEU2 (Leucine auxotrophy), Hp AUR1 (Aureobasidin auxotrophy), or Hp IMH3 (inosine monophosphate dehydrogenase auxotrophy). In some aspects, dominant markers can be: Sh-ble (Zeocin resistance), Sn-nat1 (Nourseothricin resistance), Kp-hph (Hygromycin B resistance), Tn-KanMX (G418/Geneticin resistance), or Sv-Pat (Bialaphos resistance). In some aspects, the nucleic acid construct comprises a Sc URA3 (Uracil auxotrophy) (SEQ ID NO: 5) marker.

In some aspects, the nucleic acid construct comprises a nucleic acid sequence comprising at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 5.

In some aspects, the nucleic acid construct comprises a nucleic acid sequence comprising at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 5.

“Transformation” is the term which explains the technique of exogenous DNA transfer into a selected cell resulting in a recombinant or genetically modified cell. The yeast strain transformation protocols, specifically the transformation techniques for O). polymorpha are well known in the art, and include spheroplast method, chemical transformation, electroporation, transduction, glass bead method, and biolistic particle delivery (Gellissen, et al., New yeast expression platforms based on methylotrophic O). polymorpha and Pichia pastoris and on dimorphic Arxula adeninivorans and Yarrowia lipolytica-a comparison, FEMS Yeast Res 5 (11): 1079-96 (November 2005); Sohn, et al., A family of telomere-associated autonomously replicating sequences and their functions in targeted recombination in Hansenula polymorpha DL-1,J. Bacteriol. 181:1005-1013 (1999)).

In some aspects, the O. polymorpha expressing a recombinant myoglobin gene is cultivated under known media components, physical, and operational parameters.

In some aspects, the O. polymorpha expressing a recombinant myoglobin gene is cultivated under synthetic or chemically defined media components comprising a carbon source, nitrogen source, vitamins, minerals, macro and micro nutrients, and metal supplements. The operational parameters are maintained for higher biomass and higher protein expression.

In some aspects, the recombinant O. polymorpha expressing the myoglobin gene is grown in complex media containing corn protein hydrolysates for cost-effective and commercial feasibility approaches. In some aspects, the inclusion of corn protein hydrolysate powder as the nitrogen source also supplements the production media with the necessary vitamins, minerals, and trace elements that must be added when synthetic media is used.

The term “downstream” as used herein includes the unit procedures carried out for separation of yeast biomass, cell lysis, separation and purification of heme-containing protein, specifically myoglobin from the whole cells, as the protein of interest is intracellular.

In some aspects, the downstream process for recovering the recombinant myoglobin from the O. polymorpha biomass can comprise steps (i) to (iii):
(i) Cell wash: The microbial cell biomass generated through bioreactor cultivation is harvested through bucket centrifuge (630 RS), the cell pellet is resuspended in a deionized water (also can be resuspended in a buffer, salt solution, or a suitable surfactant) and subjected to centrifugation at 100-8000 g-force or RCF. The process is repeated for 1-4 times, at each step a sample of known volume is collected for qualitative and quantitative characterization of microbial cells, metabolites, and other characteristic compounds. After the final washing step, the cells are resuspended in lysis buffer (monobasic phosphate; dibasic phosphate and/or water) for the cell disruption. In some aspects, the cell washing and recovery can be achieved through standard and well known methods like various forms of filtration and centrifugal based methods like continuous centrifugation or decantation. In some aspects, the cell lysis buffer can be changed to standard and well known buffers containing compounds like phosphate, tris, borate, citrate, acetate, glycine or diethanolamine. In some aspects, the spent media is kept with the biomass without washing.
(ii) Cell disruption: The microbial cells wild type or recombinant can be suspended in the lysis buffer and are lysed using physical (bead mill), chemical (acid, alkali, or hydrogen peroxide), enzymatic (lytic enzymes), and advanced high through-put processes (high pressure homogenization or hydrodynamic cavitation). The techniques are adopted based on the product and the preferences. For example, with the recombinant O. polymorpha expressing myoglobin, the yeast cell suspension was passed through the Dyno mill for 1-7 times. During the milling, the suspension is kept at or below 55° C. to retain the structure and functionality of the protein and heme groups for further steps. After cell disruption, the suspension is subjected to centrifugation for 60 min at 100-8000 RCF. Thereafter the supernatant is collected and may be stored frozen at −10° C. or less.
(iii) Protein recovery and purification: Final recovery and purification of myoglobin protein from the cell lysate is obtained through sequential steps of micro and ultrafiltration modules with cut-off membranes ranging from 1 μm to 1 kDa.

In some aspects, provided herein is a method describing the production of a heme-bound holoprotein. In some aspects, the method comprises the following steps: a) culturing a transgenic O. polymorpha either with homologous or heterologous nucleic acid sequence of a rate limiting gene or the sequence of genes encoding the whole heme biosynthetic pathway, operatively linked to a methanol-inducible or a constitutive promoter; b) cultivating said transgenic O. polymorpha overexpressing the heme biosynthesis without addition of an exogenous heme to the culture medium; and optionally c) isolating and purifying the heme protein. In some aspects, the expression of the recombinant animal derived heme protein is induced. In some aspects, after culturing and inducing the expression of the recombinant animal derived heme protein, the cells are harvested and spray dried. In some aspects, after culturing and inducing the expression of the recombinant animal derived heme protein, the cells are harvested, lysed and spray dried. In some aspects, after culturing and inducing the expression of the recombinant animal derived heme protein, the cells are harvested, lysed, hydrolyzed its content and spray dried.

In some aspects, provided herein is a method of producing a recombinant animal derived heme protein. In some aspects, the method comprises a) introducing a nucleic acid construct into an O. polymorpha strain, wherein said nucleic acid construct comprises a promoter operatively linked to a nucleic acid encoding an animal derived heme protein; b) culturing said transgenic cells of O. polymorpha without overexpression of the heme biosynthesis pathway or the exogenous supplementation of heme molecules to the culture medium; and optionally c) isolating the animal heme protein from the culture medium or the yeast cells based on intracellular or extracellular expression. In some aspects, the expression of the recombinant animal derived heme protein is induced. In some aspects, after culturing and inducing the expression of the recombinant animal derived heme protein, the cells are harvested and spray dried. In some aspects, after culturing and inducing the expression of the recombinant animal derived heme protein, the cells are harvested, lysed and spray dried. In some aspects, after culturing and inducing the expression of the recombinant animal derived heme protein, the cells are harvested, lysed, hydrolyzed its content and spray dried.

In some aspects, the method produces a heme-protein with at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or at least about 85%, or at least about 90%, or 100% heme loaded holo protein. In some aspects, the said heme loading on the protein can be estimated through presented HPLC quantification or calorimetric adsorption methods.

In some aspects, the method produces a heme-protein with at least 15%, at least 20%, at least 25%, at least 30%, at least 35%, at least 40%, at least 45%, at least 50%, at least 55%, at least 60%, at least 65%, at least 70%, at least 75%, or at least 80%, or at least 85%, or at least 90%, or 100% heme loaded holo protein.

In some aspects, the method produces a heme-protein with about 15%, about 20%, about 25%, about 30%, about 35%, about 40%, about 45%, about 50%, about 55%, about 60%, about 65%, about 70%, about 75%, or about 80%, or about 85%, or about 90%, or 100% heme loaded holo protein.

In some aspects, provided herein is a method of producing a recombinant bovine myoglobin, the method comprising; a) introducing a nucleic acid encoding for a recombinant bovine myoglobin operatively linked to a promoter into an O. polymorpha yeast cell; b) culturing said O. polymorpha containing said recombinant bovine myoglobin nucleic acid to promote the expression of the recombinant myoglobin neither with the overexpression of the whole heme biosynthetic pathway nor the exogenous supplementation of heme to the cultivation medium. In some aspects, the method further comprises extracting and purifying said recombinant bovine myoglobin and subjecting all remaining fractions to further processing to create hydrolyzed yeast extracts. In some aspects, the heme biosynthetic pathway in O. polymorpha may be modified or exogenous heme may be supplemented to the culture media.

In some aspects, provided herein is a method of producing a heme-protein comprising greater than 50% of heme loading. In some aspects, the method comprises the following steps: a. providing a culture of a transgenic O. polymorpha comprising a nucleic acid encoding a heme protein operatively linked to a methanol-inducible promoter; b. culturing said transgenic O. polymorpha without adding exogenous heme to the culture; and c. isolating and purifying the heme protein.

In some aspects, the method produces a heme-protein with greater than at least about 50%, at least about 55%, at least about 60%, at least about 65%, at least about 70%, at least about 75%, or at least about 80% of heme loading.

In some aspects, the method produces a heme-protein with greater than about 50%, about 55%, about 60%, about 65%, about 70%, about 75%, or about 80% of heme loading.

In some aspects, the method produces a heme-protein with greater than at least 50%, at least 55%, at least 60%, at least 65%, at least 70%, at least 75%, or at least 80% of heme loading.

In some aspects, provided herein is a method of producing an animal derived heme protein with greater than 50% of heme loading. In some aspects, the method comprises a. introducing a nucleic acid construct into an O. polymorpha strain, wherein said nucleic acid construct comprises a promoter operatively linked to a nucleic acid encoding an animal derived heme protein; b. culturing said transgenic cells of O. polymorpha without adding exogenous heme molecules to the culture; and c. isolating the animal heme protein from the culture.

In some aspects, the method produces an animal derived heme protein with greater than at least about 50%, at least about 55%, at least about 60%, at least about 65%, at least about 70%, at least about 75%, or at least about 80% of heme loading.

In some aspects, the method produces an animal derived heme protein with greater than about 50%, about 55%, about 60%, about 65%, about 70%, about 75%, or about 80% of heme loading.

In some aspects, the method produces an animal derived heme protein with greater than at least 50%, at least 55%, at least 60%, at least 65%, at least 70%, at least 75%, or at least 80% of heme loading.

In some aspects, provided herein is a method of producing a recombinant bovine myoglobin comprising greater than 50% of the recombinant bovine myoglobin loaded with heme. In some aspects, the method comprises a. introducing a nucleic acid encoding for a recombinant bovine myoglobin operatively linked to a promoter into an O. polymorpha yeast cell; b. culturing said O. polymorpha containing said recombinant bovine myoglobin nucleic acid to promote the expression of the recombinant myoglobin without adding heme to the culture, thus expressing the recombinant myoglobin. In some aspects, the method further comprises extracting and purifying said recombinant bovine myoglobin.

In some aspects, the method produces a recombinant myoglobin comprising greater than 50% of the recombinant bovine myoglobin loaded with heme. In some aspects, the method produces a recombinant myoglobin comprising at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or at least about 85%, or at least about 90%, or 100% of the recombinant bovine myoglobin loaded with heme.

In some aspects, the method produces a recombinant myoglobin comprising about 50%, about 55%, about 60%, about 65%, about 70%, about 75%, or about 80% of the recombinant bovine myoglobin loaded with heme.

In some aspects, the method produces a recombinant myoglobin comprising at least 50%, at least 55%, at least 60%, at least 65%, at least 70%, at least 75%, or at least 80% of the recombinant bovine myoglobin loaded with heme.

In some aspects, the transgenic O. polymorpha does not comprise an exogenous transcriptional activator of the heme biosynthetic pathway or an exogenous component of the heme biosynthetic pathway.

In some aspects, the heme protein is selected from the group consisting of animal derived heme protein and plant derived heme protein.

In some aspects, said animal derived heme protein is selected from the group consisting of bovine derived heme protein, porcine derived heme protein, ovine derived heme protein, equine derived heme protein, and caprine derived heme protein.

In some aspects, said animal derived heme protein is selected from the group consisting of: hemoglobin, myoglobin, neuroglobin, cytoglobin, cytochrome P450s, cytochrome c oxidase, ligninases, catalase, peroxidases, cytochrome a, cytochrome b, and cytochrome c.

In some aspects, said nucleic acid comprises a nucleic acid sequence comprising at least about 70% identity to the nucleic acid sequence of SEQ ID NO: 1.

In some aspects, said nucleic acid comprises a nucleic acid sequence comprising at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to the nucleic acid sequence of SEQ ID NO: 1.

In some aspects, said nucleic acid comprises a nucleic acid sequence comprising at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence of SEQ ID NO: 1.

In some aspects, the myoglobin comprises an amino acid sequence corresponding to SEQ ID NO: 6. In some aspects, the myoglobin comprises an amino acid sequence comprising at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to the amino acid sequence of SEQ ID NO: 6. In some aspects, the myoglobin comprises an amino acid sequence comprising at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 6.

In some aspects, provided herein is a food composition comprising any of the heme proteins disclosed herein or any of the bovine myoglobin proteins disclosed herein.

In some aspects, provided herein is a meat analog food composition comprising a lysate of any of the cultured O. polymorpha provided herein.

In some aspects, provided herein is a meat analog food composition comprising any of the heme proteins disclosed herein or any of the bovine myoglobin proteins disclosed herein.

The recombinant heme proteins can be isolated to be used to prepare food compositions. In some aspects, the recombinant heme proteins produced by the modified O. polymorpha may be used in its entirety, fractions and modifications thereof including solubilized, precipitated, partially or fully hydrolyzed, cross-linked, emulsified, texturized, cooked, extruded, high-shear-mixed, couette cell produced, reacted (including nitrite based curing) structured versions to prepare meat and meat-like (i.e., meat analogs) food stuffs including comminuted meat and meat-analogues such as minced meat, meat strips, chunks, cubes and steaks; reconstituted, 3D-printed, 3D-printed and formed meat-like products including burgers, filets, balls, rings, fingers, rings, fingers, sticks, slabs; reconstituted and stuffed/filled meat-like (i.e., meat analog) products including sausages, ham-like products, spreadables, reconstituted and coated meat-like products including nuggets, patties, strips, poppers, rings and more.

### O. Polymorpha Derived Extracts.

In some aspects, provided herein is a cell extract derived from a culture of O. polymorpha. O. polymorpha is a methylotrophic yeast that is a good source of molecules for the food and nutrition industry.

In some aspects, the O. polymorpha extract is obtained from a genetically modified O. polymorpha producing a recombinant heme protein. Said recombinant heme protein can be a recombinant plant heme protein, recombinant bacterial heme protein, recombinant fungal heme protein, or a recombinant animal heme protein. In some aspects, the recombinant protein is selected from the group consisting of: hemoglobin, myoglobin, leghemoglobin, neuroglobin, cytoglobin, cytochrome P450s, cytochrome c oxidase, ligninase, catalase, peroxidases, cytochrome a, cytochrome b, and cytochrome c.

In some aspects, the O. polymorpha extract comprises at least 0.03% w/w, at least 0.05% w/w, at least 0.1% w/w, at least 0.15% w/w, at least 0.2% w/w, at least 0.225% w/w, at least 0.25% w/w, at least 0.275% w/w, at least 0.30% w/w, at least 0.4% w/w, at least 0.5% w/w, at least 0.6% w/w, at least 0.7% w/w, at least 0.8% w/w, at least 0.9% w/w, at least 1.0% w/w, at least 1.1% w/w, at least 1.2% w/w, at least 1.3% w/w, at least 1.4% w/w, at least 1.5% w/w, at least 1.6% w/w, at least 1.7% w/w, at least 1.8% w/w, at least 1.9% w/w, at least 2.0% w/w, at least 2.1% w/w, at least 2.2% w/w, at least 2.3% w/w, at least 2.4% w/w, at least 2.5% w/w, at least 2.6% w/w, at least 2.7% w/w, at least 2.8% w/w, at least 2.9% w/w, at least 3.0% w/w, or at least 3.1% w/w of heme.

In some aspects, the O. polymorpha extract comprises about 0.03% w/w, about 0.05% w/w, about 0.1% w/w, about 0.15% w/w, about 0.2% w/w, about 0.225% w/w, about 0.25% w/w, about 0.275% w/w, about 0.30% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, or about 3.1% w/w of heme.

In some aspects, the O. polymorpha extract comprises from about 0.03% w/w to about 3.1% w/w, from about 0.1% w/w to about 3.1% w/w, from about 0.5% w/w to about 3.1% w/w, from about 1% w/w to about 3.1% w/w, from about 1.5% w/w to about 3.1% w/w, from about 2% w/w to about 3.1% w/w, from about 2.5% w/w to about 3.1% w/w, from about 0.1% w/w to about 3% w/w, from about 0.1% w/w to about 2.5% w/w, from about 0.1% w/w to about 2% w/w, from about 0.1% w/w to about 1.5% w/w, from about 0.1% w/w to about 1% w/w, or from about 0.1% w/w to about 0.5% w/w of heme.

In some aspects, the O. polymorpha extract comprises at least 0.8% w/w of glutamic acid, and at least 0.2% glutathione.

In some aspects, the O. polymorpha extract comprises at least 0.8% w/w, at least 0.9% w/w, at least 1% w/w, at least 1.5% w/w, at least 2% w/w, at least 2.5% w/w, at least 3% w/w, at least 3.5% w/w, or at least 4% w/w of glutamic acid.

In some aspects, the O. polymorpha extract comprises about 0.8% w/w, about 0.9% w/w, about 1% w/w, about 1.5% w/w, about 2% w/w, about 2.5% w/w, about 3% w/w, about 3.5% w/w, or about 4% w/w of glutamic acid.

In some aspects, the O. polymorpha extract comprises from about 0.8% w/w to about 4% w/w, from about 1% w/w to about 4% w/w, from about 1.5% w/w to about 4% w/w, from about 2% w/w to about 4% w/w, from about 2.5% w/w to about 4% w/w, from about 3% w/w to about 4% w/w, from about 3.5% w/w to about 4% w/w, from about 0.8% w/w to about 3.5% w/w, from about 0.8% w/w to about 3% w/w, from about 0.8% w/w to about 2.5% w/w, from about 0.8% w/w to about 2% w/w, from about 0.8% w/w to about 1.5% w/w, or from about 0.8% w/w to about 1% w/w of glutamic acid.

In some aspects, the O. polymorpha extract comprises at least 0.2%, at least 0.3%, at least 0.4%, at least 0.5%, at least 0.6%, at least 0.7%, at least 0.8%, at least 0.9%, or at least 1% of glutathione.

In some aspects, the O. polymorpha extract comprises about 0.2%, about 0.3%, about 0.4%, about 0.5%, about 0.6%, about 0.7%, about 0.8%, about 0.9%, or about 1% of glutathione.

In some aspects, the O. polymorpha extract comprises from about 0.2% to about 1%, from about 0.5% to about 1%, from about 0.7% to about 1%, or from about 0.2% to about 0.5% of glutathione.

In some aspects, the O. polymorpha extract is rich in 5′ ribonucleotides, such as 5′ GMP and/or 5′ IMP. In some aspects, the O. polymorpha extract comprises at least 0.007% of 5′ ribonucleotides. In some aspects, the O. polymorpha extract comprises at least 0.007% of 5′ GMP.

In some aspects, the O. polymorpha extract comprises at least 0.005%, at least 0.006%, at least 0.007%, at least 0.01%, at least 0.02%, at least 0.03%, at least 0.04%, or at least 0.05%, of 5′ ribonucleotides.

In some aspects, the O. polymorpha extract comprises about 0.005%, about 0.006%, about 0.007%, about 0.01%, about 0.02%, about 0.03%, about 0.04%, or about 0.05%, of 5′ ribonucleotides.

In some aspects, the O. polymorpha extract comprises from about 0.005% to about 0.05%, from about 0.007% to about 0.05%, from about 0.01% to about 0.05%, from about 0.02% to about 0.05%, from about 0.03% to about 0.05%, from about 0.04% to about 0.05%, from about 0.007% to about 0.04%, from about 0.007% to about 0.03%, from about 0.007% to about 0.02%, or from about 0.007% to about 0.01% of 5′ ribonucleotides.

In some aspects, the O. polymorpha extract comprises at least 0.005%, at least 0.006%, at least 0.007%, at least 0.01%, at least 0.02%, at least 0.03%, at least 0.04%, or at least 0.05%, of 5′ GMP.

In some aspects, the O. polymorpha extract comprises about 0.005%, about 0.006%, about 0.007%, about 0.01%, about 0.02%, about 0.03%, about 0.04%, or about 0.05%, of 5′ GMP.

In some aspects, the O. polymorpha extract comprises from about 0.005% to about 0.05%, from about 0.007% to about 0.05%, from about 0.01% to about 0.05%, from about 0.02% to about 0.05%, from about 0.03% to about 0.05%, from about 0.04% to about 0.05%, from about 0.007% to about 0.04%, from about 0.007% to about 0.03%, from about 0.007% to about 0.02%, or from about 0.007% to about 0.01% of 5′ GMP.

In some aspects, the O. polymorpha extract comprises at least 0.005%, at least 0.006%, at least 0.007%, at least 0.01%, at least 0.02%, at least 0.03%, at least 0.04%, or at least 0.05%, of 5′ IMP.

In some aspects, the O. polymorpha extract comprises about 0.005%, about 0.006%, about 0.007%, about 0.01%, about 0.02%, about 0.03%, about 0.04%, or about 0.05%, of 5′ IMP.

In some aspects, the O. polymorpha extract comprises from about 0.005% to about 0.05%, from about 0.007% to about 0.05%, from about 0.01% to about 0.05%, from about 0.02% to about 0.05%, from about 0.03% to about 0.05%, from about 0.04% to about 0.05%, from about 0.007% to about 0.04%, from about 0.007% to about 0.03%, from about 0.007% to about 0.02%, or from about 0.007% to about 0.01% of 5′ IMP.

In some aspects, the O. polymorpha extract comprises at least: 0.03% w/w of heme, 0.8% w/w of glutamic acid and 0.2% w/w of glutathione.

In some aspects, the O. polymorpha extract comprises at least: 0.03% w/w of heme, 0.8% w/w of glutamic acid, 0.2% w/w of glutathione, and 0.007% of 5′ ribonucleotides, (e.g., 5′ IMP and/or 5′ GMP).

In some aspects, the O. polymorpha extract comprises at least: 0.03% w/w of heme, 0.8% w/w of glutamic acid, 0.2% w/w of glutathione, and 0.007% of 5′ GMP.

In some aspects, the O. polymorpha extract comprises at least 0.03% w/w of heme and at least 0.007% of 5′ GMP.

In some aspects, the O. polymorpha extract comprises at least 0.8% w/w of glutamic acid, 0.2% of glutathione, and 0.007% of 5′-ribonucleotides, and substantially no heme content.

In some aspects, the O. polymorpha extract comprises about 0.8% w/w of glutamic acid, and about 0.2% glutathione.

In some aspects, the O. polymorpha extract is rich in 5′ ribonucleotides, such as 5′ GMP and/or 5′ IMP. In some aspects, the O. polymorpha extract comprises about 0.007% of 5′ ribonucleotides. In some aspects, the O. polymorpha extract comprises about 0.007% of 5′ GMP.

In some aspects, the O. polymorpha extract comprises about 0.03% w/w of heme, about 0.8% w/w of glutamic acid and about 0.2% w/w of glutathione.

In some aspects, the O. polymorpha extract comprises about 0.03% w/w of heme, about 0.8% w/w of glutamic acid, about 0.2% w/w of glutathione, and about 0.007% of 5′ ribonucleotides, (e.g., 5′ IMP and/or 5′ GMP).

In some aspects, the O. polymorpha extract comprises about 0.03% w/w of heme, about 0.8% w/w of glutamic acid, about 0.2% w/w of glutathione, and about 0.007% of 5′ GMP

In some aspects, the O. polymorpha extract comprises about 0.03% w/w of heme and about 0.007% of 5′ GMP.

In some aspects, the O. polymorpha extract comprises about 0.8% w/w of glutamic acid, about 0.2% of glutathione, and about 0.007% of 5′-ribonucleotides, and substantially no heme content.

In some aspects, the method to obtain an O. polymorpha extract comprises the steps of: i) culturing and propagating the O. polymorpha cells, ii) optionally washing and harvesting the cells; and iii) lysing the cells. The cell lysing may be done with known methods in the art, such as, autolysis, physical lysis, mechanical lysis, chemical lysis (mediated with acids or alkaline solutions), enzymatic lysis, microbial lysis, or a combination of thereof.

In some aspects, after the culturing and propagation of the O. polymorpha cells, the cells are washed and harvested, to create an O. polymorpha biomass.

In some aspects, after the culturing and propagation of the O. polymorpha cells, the cells are collected with the culture media, or with a part of the culture media, to create an O. polymorpha biomass.

In some aspects, after the lysis, in order to create a solid O. polymorpha biomass, it is spray dried. In some aspects, after the lysis, in order to create a solid O. polymorpha extract, it is spray dried.

In some aspects, in order to obtain an extract rich in heme the temperature is kept low, due to the instability of the heme-molecule and its rapid degradation at higher temperatures. In some aspects, the temperature of the method to obtain the O. polymorpha extract rich in heme, is less than 70° C. In some aspects, the temperature of the method to obtain the O. polymorpha extract rich in heme is from about 40° C. to about 70° C. In some aspects, the temperature of the method to obtain the O. polymorpha extract rich in heme is from about 45° C. to about 60° C. In some aspects, the temperature of the method to obtain the O. polymorpha extract rich in heme is from about 48° C. to about 58° C. In some aspects, the temperature of the method to obtain the O. polymorpha extract rich in heme is from about 49° C. to about 57° C. In some aspects, the temperature of the method to obtain the O. polymorpha extract rich in heme is from about 50° C. to about 55° C. If a thermal pasteurization step is needed, the temperature of the pasteurization step can be up to about 90° C. from about 1 to about 100 seconds.

In some aspects, the temperature of the method to obtain an O. polymorpha extract is independent of the temperature, wherein the heme has been previously isolated and separated from the biomass.

In some aspects, the method to obtain an O. polymorpha extract is independent of the temperature, whereas the resulting extract comprises substantially no heme content.

In some aspects, the O. polymorpha biomass contains at least 0.03% w/w heme per cell dry weight.

In some aspects, the O. polymorpha extract contains at least 0.03% w/w heme per dry weight.

In some aspects, the O. polymorpha biomass contains about 0.03% w/w heme per cell dry weight.

In some aspects, the O. polymorpha extract contains about 0.03% w/w heme per dry weight.

In some aspects, in order to obtain an O. polymorpha extract with high-glutamic derived compounds, the proteins of the O. polymorpha extract are hydrolyzed, and the enzymatic conversion of glutamic acids into glutamate using a glutaminase are further induced.

In some aspects, to obtain an O. polymorpha extract with high 5′ ribonucleotides, the nucleic acids are hydrolyzed preferably with a nuclease or chemicals. In some aspects, to produce 5′ IMP, through the conversion of 5′ AMP to 5′ IMP, the addition of an enzyme such as the 5′ Adenylic deaminase is needed. To achieve the highest amount 5′ ribonucleotides is important to inactivate an O. polymorpha endogenous acid, neutral or alkaline phosphatase. Endogenous phosphatases can hydrolyze the phosphate groups from the nucleotides producing nucleosides, the latter of which are no longer of interest for flavoring. In some aspects, the O. polymorpha extract is rich in 5′ GMP and/or 5′ IMP.

In some aspects, the method to obtain a hydrolyzed O. polymorpha extract rich in heme and/or glutamic derived compounds and rich in 5′ ribonucleotides, the temperature of the method is above 70° C. In some aspects, the temperature of the method to obtain the hydrolyzed O. polymorpha extract rich in heme is from about 40° C. to about 70° C. In some aspects, the temperature of the method to obtain the hydrolyzed O. polymorpha extract rich in heme is from about 45° C. to about 60° C. In some aspects, the temperature of the method to obtain the hydrolyzed O. polymorpha extract rich in heme is from about 48° C. to about 58° C. In some aspects, the temperature of the method to obtain the hydrolyzed O. polymorpha extract rich in heme is from about 49° C. to about 57° C. In some aspects, the temperature of the method to obtain the hydrolyzed O. polymorpha extract rich in heme is from about 50° C. to about 55° C. If a thermal pasteurization step is needed, the temperature of the pasteurization step can be up to about 90° C. from about 1 to about 100 seconds.

In some aspects, the temperature of the method to obtain a hydrolyzed O. polymorpha extract is independent of the temperature, wherein the heme has been previously isolated and separated from the extract.

In some aspects, the temperature of the method to obtain a hydrolyzed O. polymorpha extract is independent of the temperature, whereas the resulting hydrolyzed extract comprises substantially no heme content.

In some aspects, the lysis of the O. polymorpha cells is achieved at alkaline pH from about 7 to about 12, from about 7.5 to about 10.5, from about 8 to about 10, or from about 8.5 to about 9.0. At the pH disclosed herein, the lysis of the cell is achieved but also the deactivation of the endogenous acid phosphatase and it keeps the viscosity low enough for proper stirring during the process.

In some aspects, the lysis of the O. polymorpha cell at alkaline pH is incubated with a lytic enzyme cocktail containing proteases (preferably proteases which are active in a neutral to alkaline pH range), glucanases (preferably glucanases which are active in a neutral to alkaline pH range), mannanases (preferably active in a neutral to alkaline pH range), and chitinases (which are active in a neutral to alkaline pH range). Some of the well-known commercial lytic enzymes available are Alcalase®, Esperase®, Savinase®, Neutrase®, Kitalase, Zymolyase, Lyticase, Glusulase, Promod® (Sharma, M., et al., A review on microbial alkaline protease: an essential tool for various industrial approaches, Industrial Biotechnology, 15 (2): 69-78 (2019); Sharma, K. M. et al., Microbial alkaline proteases: Optimization of production parameters and their properties, Journal of Genetic Engineering and Biotechnology, 15 (1): 115-126 (2017), strem.com/uploads/technical_notes/06-3115tech.pdf).

In some aspects, a proteolysis with an alkaline lytic cocktail can be further pursued with other proteases and adjusting the pH of the media to suit the activity of the chosen protease. After lowering the pH to neutral or even acid (pH from about 4 to about 7) the action of neutral or acid proteolytic enzymes can be made. In some aspects, proteolysis can be achieved by a mixture of proteolytic enzymes. Proteolytic enzymes commercially available such as Protease AR, Protease P, ProteAX, Flavourzyme®, Umamizyme®, Papain, Bromelain, Protamex®, Neutrase® and Sumizyme BNP-L can be used.

In some aspects, after the proteolysis, the hydrolyzed O. polymorpha extract contains a majority of small peptides, e.g., below 1.0 kDa.

In some aspects, after the proteolysis, a conversion of glutamine into glutamic acid or its salt is used to enrich the content of glutamic compounds that are well known as conferring umami taste. In some aspects, the conversion can be achieved using glutaminase (e.g., L-glutaminase from Escherichia coli, glutaminase from Bacillus amyloliquefaciens, glutaminase from Bacillus licheniformis, protein-glutaminase (PG), from Chryseobacterium proteolyticum).

In some aspects, the proteolyzed O. polymorpha extract is further treated with deaminases comprising at least 0.8% of glutamic acid.

In some aspects, the proteolyzed O. polymorpha extract is further treated with deaminases comprising at least 0.8% glutathione.

In some aspects, the nucleic acid hydrolysis and AMP conversion into IMP can be performed after or before the proteolysis.

Flavor precursor mixtures.

A flavor precursor mixture is a mixture of compounds characterized by providing flavors, taste, aroma to food products. The flavors and aroma are released by cooking the mixture or by subjecting the mixture to a Maillard reaction.

In some aspects, the flavor precursor mixture comprises an O. polymorpha extract. In some aspects, the flavor precursor mixture comprises a hydrolyzed O. polymorpha extract.

In some aspects, the hydrolyzed O. polymorpha extract is filtered to retain soluble molecules with a size equal or smaller than 30 kDa. In some aspects, the hydrolyzed O. polymorpha extract is filtered to retain soluble molecules with a size equal or smaller than 25 kDa. In some aspects, the hydrolyzed O. polymorpha extract is filtered to retain soluble molecules with a size equal or smaller than 20 kDa. In some aspects, the hydrolyzed O. polymorpha extract is filtered to retain soluble molecules with a size equal or smaller than 15 kDa. In some aspects, the hydrolyzed O. polymorpha extract is filtered to retain soluble molecules with a size equal or smaller than 10 kDa. In some aspects, the hydrolyzed O. polymorpha extract is filtered to retain soluble molecules with a size equal or smaller than 5 kDa. In some aspects, the hydrolyzed O. polymorpha extract is filtered to retain soluble molecules with a size equal or smaller than 3 kDa. In some aspects, the hydrolyzed O. polymorpha extract is filtered to retain soluble molecules with a size equal or smaller than 2 kDa. In some aspects, the hydrolyzed O. polymorpha extract is filtered to retain soluble molecules with a size equal or smaller than 1 kDa. In some aspects, the hydrolyzed O. polymorpha extract is filtered to retain soluble molecules with a size equal or smaller than 0.5 kDa. In some aspects, the hydrolyzed O. polymorpha extract is filtered to retain insoluble molecules.

In some aspects, the O. polymorpha biomass, extract and/or hydrolyzed extract of the flavor precursor mixture comprises at least 0.03% w/w, at least 0.05% w/w, at least 0.1% w/w, at least 0.15% w/w, at least 0.2% w/w, at least 0.225% w/w, at least 0.25% w/w, at least 0.275% w/w, at least 0.30% w/w, at least 0.4% w/w, at least 0.5% w/w, at least 0.6% w/w, at least 0.7% w/w, at least 0.8% w/w, at least 0.9% w/w, at least 1.0% w/w, at least 1.1% w/w, at least 1.2% w/w, at least 1.3% w/w, at least 1.4% w/w, at least 1.5% w/w, at least 1.6% w/w, at least 1.7% w/w, at least 1.8% w/w, at least 1.9% w/w, at least 2.0% w/w, at least 2.1% w/w, at least 2.2% w/w, at least 2.3% w/w, at least 2.4% w/w, at least 2.5% w/w, at least 2.6% w/w, at least 2.7% w/w, at least 2.8% w/w, at least 2.9% w/w, at least 3.0% w/w, or at least 3.1% w/w of heme.

In some aspects, the O. polymorpha biomass, extract and/or hydrolyzed extract of the flavor precursor mixture comprises about 0.03% w/w, about 0.05% w/w, about 0.1% w/w, about 0.15% w/w, about 0.2% w/w, about 0.225% w/w, about 0.25% w/w, about 0.275% w/w, about 0.30% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, or about 3.1% w/w of heme.

In some aspects, the O. polymorpha biomass, extract and/or hydrolyzed extract of the flavor precursor mixture comprises at least 0.8% w/w of glutamic acid. In some aspects, the O. polymorpha biomass, extract and/or hydrolyzed extract of the flavor precursor mixture comprises at least 0.2% w/w of glutathione. In some aspects, the O. polymorpha biomass, extract and/or hydrolyzed extract of the flavor precursor mixture comprises at least 0.007% of 5′ GMP. In some aspects, the O. polymorpha biomass, extract and/or hydrolyzed extract of the flavor precursor mixture comprises at least 0.03% w/w of heme and/or 0.8% w/w of glutamic acid and/or 0.2% w/w of glutathione and/or 0.007% of 5′ GMP.

In some aspects, the O. polymorpha biomass, extract and/or hydrolyzed extract of the flavor precursor mixture comprises about 0.8% w/w of glutamic acid. In some aspects, the O. polymorpha biomass, extract and/or hydrolyzed extract of the flavor precursor mixture comprises about 0.2% w/w of glutathione. In some aspects, the O. polymorpha biomass, extract and/or hydrolyzed extract of the flavor precursor mixture comprises about 0.007% of 5′ GMP. In some aspects, the O. polymorpha biomass, extract and/or hydrolyzed extract of the flavor precursor mixture comprises about 0.03% w/w of heme, about 0.8% w/w of glutamic acid, about 0.2% w/w of glutathione, and/or about 0.007% of 5′ GMP.

In some aspects, the content of the O. polymorpha biomass, extract and/or hydrolyzed extract in the mixture to obtain a flavor precursor mixture, is at least 0.01% of the mixture. In some aspects, the content of the O. polymorpha biomass, extract and/or hydrolyzed extract in the mixture to obtain a flavor precursor mixture, is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, or at least 90% of the mixture.

In some aspects, the content of the O. polymorpha biomass, extract and/or hydrolyzed extract in the mixture to obtain a flavor precursor mixture, is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% of the mixture.

In some aspects, the content of the O. polymorpha biomass, extract and/or hydrolyzed extract in the mixture to obtain a flavor precursor mixture, is from about 1% to about 90%, from about 1% to about 80%, from about 1% to about 70%, from about 1% to about 60%, from about 1% to about 50%, from about 1% to about 40%, from about 1% to about 30%, from about 1% to about 20%, from about 1% to about 10%, from about 1% to about 5%, from about 5% to about 90%, from about 10% to about 90%, from about 20% to about 90%, 20% to about 72%, from about 30% to about 90%, from about 40% to about 90%, from about 50% to about 90%, from about 60% to about 90%, from about 70% to about 90%, or from about 80% to about 90% of the mixture.

In some aspects, the O. polymorpha biomass, extract and/or hydrolyzed extract flavor precursor mixture further comprises monosaccharides such as pentoses and hexoses. The monosaccharides can be reducing sugars selected from the group consisting of: Rhamnose, Arabinose, Ribose, D-Xylose, Glucose, Glucosamine, Galactose, Fructose, steviols, and synthetic sweeteners.

In some aspects, the O. polymorpha biomass, extract and/or hydrolyzed extract flavor precursor mixture comprises at least 0.1% w/w, at least 0.2% w/w, at least 0.3% w/w, at least 0.4% w/w, at least 0.5% w/w, at least 0.6% w/w, at least 0.7% w/w, at least 0.8% w/w, at least 0.9% w/w, at least 1% w/w, at least 1.5% w/w, at least 2% w/w, at least 2.5% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, at least 15% w/w, at least 20% w/w, at least 25% w/w, at least 30% w/w, or at least 35% w/w of monosaccharides.

In some aspects, the O. polymorpha biomass, extract and/or hydrolyzed extract flavor precursor mixture comprises about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.5% w/w, about 2% w/w, about 2.5% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 20% w/w, about 25% w/w, about 30% w/w, or about 35% w/w of monosaccharides.

In some aspects, the O. polymorpha biomass, extract and/or hydrolyzed extract flavor precursor mixture comprises from about 0.1% w/w to about 35% w/w, from about 0.1% w/w to about 30% w/w, from about 0.1% w/w to about 25% w/w, from about 0.1% w/w to about 20% w/w, from about 0.1% w/w to about 15% w/w, from about 0.1% w/w to about 10% w/w, from about 0.1% w/w to about 5% w/w, from about 0.1% w/w to about 1% w/w, from about 1% w/w to about 35% w/w, from about 5% w/w to about 35% w/w, from about 10% w/w to about 35% w/w, from about 15% w/w to about 35% w/w, from about 20% w/w to about 35% w/w, from about 25% w/w to about 35% w/w, or from about 30% w/w to about 35% w/w, of monosaccharides.

In some aspects, the O. polymorpha biomass, extract and/or hydrolyzed extract flavor precursor mixture further comprises disaccharides such as sucrose, lactose and maltose.

In some aspects, the O. polymorpha biomass, extract and/or hydrolyzed extract flavor precursor mixtures comprise amino acids such as alanine, arginine, cysteine, glycine, histidine, isoleucine, leucine, lysine, methionine, proline, serine, threonine, tryptophan, tyrosine, valine, glutamine, and the mixture thereof. In some aspects, the flavor precursor mixture comprises di, tri, tetra, penta, hexa, hepta, octa, nona, decapeptides. In some aspects, the flavor precursor mixture comprises oligopeptides. In some aspects, the O. polymorpha biomass, extract and/or hydrolyzed extract flavor precursor mixture comprises glutathione.

In some aspects, polymorpha biomass, extract and/or hydrolyzed extract flavor precursor mixtures comprise at least 0.4% w/w of histidine.

In some aspects, polymorpha biomass, extract and/or hydrolyzed extract flavor precursor mixtures comprise at least 0.4% w/w, at least 0.5% w/w, at least 0.6% w/w, at least 0.7% w/w, at least 0.8% w/w, at least 0.9% w/w, at least 1% w/w, at least 1.5% w/w, at least 2% w/w, at least 2.5% w/w, or at least 3% w/w of histidine.

In some aspects, polymorpha biomass, extract and/or hydrolyzed extract flavor precursor mixtures comprise about 0.4% w/w, about 0.5% w/w, about 0.6% w/w, about 0.7% w/w, about 0.8% w/w, about 0.9% w/w, about 1% w/w, about 1.5% w/w, about 2% w/w, about 2.5% w/w, or about 3% w/w of histidine.

In some aspects, polymorpha biomass, extract and/or hydrolyzed extract flavor precursor mixtures comprise from about 0.4% w/w to about 3% w/w, from about 0.5% w/w to about 3% w/w, from about 1% w/w to about 3% w/w, from about 1.5% w/w to about 3% w/w, from about 2% w/w to about 3% w/w, from about 2.5% w/w to about 3% w/w, from about 0.4% w/w to about 2.5% w/w, from about 0.4% w/w to about 2% w/w, from about 0.4% w/w to about 1.5% w/w, or from about 0.4% w/w to about 1% w/w of histidine.

In some aspects, polymorpha biomass, extract and/or hydrolyzed extract flavor precursor mixtures comprise at least 15% w/w of amino acids.

In some aspects, polymorpha biomass, extract and/or hydrolyzed extract flavor precursor mixtures comprise at least 0.1% w/w, at least 0.2% w/w, at least 0.3% w/w, at least 0.4% w/w, at least 0.5% w/w, at least 0.6% w/w, at least 0.7% w/w, at least 0.8% w/w, at least 0.9% w/w, at least 1% w/w, at least 1.5% w/w, at least 2% w/w, at least 2.5% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, or at least 15% w/w of amino acids.

In some aspects, polymorpha biomass, extract and/or hydrolyzed extract flavor precursor mixtures comprise about 0.4% w/w, about 0.5% w/w, about 0.6% w/w, about 0.7% w/w, about 0.8% w/w, about 0.9% w/w, about 1% w/w, about 1.5% w/w, about 2% w/w, about 2.5% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, or about 15% w/w of amino acids.

In some aspects, polymorpha biomass, extract and/or hydrolyzed extract flavor precursor mixtures comprise from about 0.1% w/w to about 15% w/w, from about 0.5% w/w to about 15% w/w, from about 1% w/w to about 15% w/w, from about 5% w/w to about 15% w/w, from about 10% w/w to about 15% w/w, from about 0.1% w/w to about 10% w/w, from about 0.1% w/w to about 5% w/w, from about 0.1% w/w to about 4% w/w, from about 0.1% w/w to about 3% w/w, or from about 0.1% w/w to about 1% w/w of amino acids.

In some aspects, the O. polymorpha biomass, extract and/or hydrolyzed extract flavor precursor mixtures comprises vitamins such as vitamin A, vitamin C, vitamin D, vitamin E, vitamin B, vitamin K and the mixture thereof. In some aspects, the vitamin is a Vitamin B complex. In some aspects, the vitamin is a vitamin B1.

In some aspects, the O. polymorpha biomass, extract and/or hydrolyzed extract flavor precursor mixture further comprises Saccharomyces cerevisiae, Cyberlindnera jadinii, seaweed/kelp and/or mushroom-derived extracts.

In some aspects, the O. polymorpha biomass, extract and/or hydrolyzed extract flavor precursor mixture further comprises a fat. The fat can be vegetable oil, or fatty acids. In some aspects, the fatty acids are saturated fatty acids. In some aspects, the fatty acids are unsaturated fatty acids. In some aspects, the fat is a mixture of unsaturated fatty acids and saturated fatty acids. In some aspects, the fat is an edible vegetable oil such as olive oil, canola oil, sunflower oil, soybean oil, safflower oil, chia oil, rapeseed oil, peanut oil, linseed oil, coconut oil, palm oil, and a mixture thereof.

In some aspects, the vegetable fat can be saturated fatty acids such as Lauric acid, Myristic acid, Pamitic acid, Stearic acid.

In some aspects, the vegetable fat can be monounsaturated fatty acids such as Myristoleic acid, Palmitoleic acid, cis-Vaccenic acid, Vaccenic acid, Paullinic acid, Oleic acid, Elaidic acid, 11-Eicosenoic acid, Erucic acid, Brassidic acid, Nervonic acid, Sapienic acid, Gadoleic acid, Petroselinic acid.

In some aspects, the vegetable fat can be free polyunsaturated fatty acids (PUFAs) such as Hexadecatrienoic acid (HTA), Alpha-linolenic acid (ALA), Stearidonic acid (SDA), Eicosatrienoic acid (ETE), Eicosatetraenoic acid (ETA), Eicosapentaenoic acid (EPA, Timnodonic acid), Heneicosapentaenoic acid (HPA), Docosapentaenoic acid (DPA, Clupanodonic acid), Docosahexaenoic acid (DHA, Cervonic acid), Tetracosapentaenoic acid, Tetracosahexaenoic acid (Nisinic acid), Linoleic acid (LA), Gamma-linolenic acid (GLA), Eicosadienoic acid, Dihomo-gamma-linolenic acid (DGLA), Arachidonic acid (AA), Docosadienoic acid, Adrenic acid (AdA), Docosapentaenoic acid (DPA), Tetracosatetraenoic acid, Tetracosapentaenoic acid and mixtures thereof.

In some aspects, the fat is a mixture of saturated, monounsaturated and polyunsaturated fatty acids (PUFAs).

In some aspects, the fat is a mixture of vegetable oils and cultivated fat, whereas cultivated fat is a culture of adipocytes.

In some aspects, the O. polymorpha biomass, extract and/or hydrolyzed extract flavor precursor mixture further comprises a polypeptide, protein or protein fragment. In some aspects, the polypeptide, protein or protein fragment is from a plant. In some aspects, the polypeptide, protein or protein fragment is from a fungus. In some aspects, the polypeptide, protein or protein fragment is from an animal. In some aspects, the polypeptide, protein or protein fragment is a recombinant protein. In some aspects, the polypeptide, protein or protein fragment is a recombinant heme-protein. In some aspects, the polypeptide, protein or protein fragment is a recombinant animal heme-protein.

In some aspects, the O. polymorpha biomass, extract and/or hydrolyzed extract flavor precursor mixture further comprises a carrier material. The carrier material can be a polysaccharide such as starch, modified starch, carrageenan, acacia gum, arabic gum, carboxymethyl cellulose, chitosan, and mixtures thereof. Other carriers can be S. cerevisiae extracts and salts.

### Maillard Reaction Product.

In some aspects, provided herein is a method to produce a flavor composition derived from the flavor precursors mixture.

In some aspects, the method to produce a flavor composition comprises the incubation of the flavor precursors mixture under conditions of temperature and water content to obtain the flavor composition.

In some aspects, the flavor precursors mixture is added to a food matrix and the flavor is produced during the cooking process.

A Maillard Reaction (MR) is a well-known process, wherein a Maillard Reaction Product (MRP) is the product derived from the MR. The MRP is a flavor composition that can be added to food products. The flavor could be improved when said MRP is combined with another MRP obtained by employing other kinds of amino acids or with vegetable protein hydrolysate.

In some aspects, the O. polymorpha biomass, extract and/or hydrolyzed extract flavor precursor mixture is thermally reacted, to obtain an O. polymorpha-derived MRP.

In some aspects, the temperature of the MR is within the range of from about 85° C. to about 200° C., more preferably from about 90° C. to about 160° C., most preferably from about 95° C. to about 140° C. In some aspects, the flavor precursors mixture is heated for a time period of from about 10 minutes to about 60 minutes, from about 60 minutes to about 1200 minutes, from about 120 minutes to about 900 minutes, from about 180 minutes to about 720 minutes, from about 240 minutes to about 600 minutes, or from about 300 minutes to about 540 minutes. Preferably, the pressure for the Maillard reaction is from about 0.1 to about 10 bar, from about 0.25 to about 7.5 bar, from about 0.4 to about 5 bar, from about 0.5 to about 3 bar, from about 0.75 to about 2.5 bar, from about 1 to about 2 bar. Preferably the pH for the Maillard reaction is from about 0.5 to about 11, from about 1 to about 10.5, from about 2 to about 10, from about 3 to about 9, from about 4 to about 8.5, or from about 5 to about 8.

In some aspects, the temperature of the MR is within the range of from 85° C. to 200° C., more preferably from 90° C. to 160° C., most preferably from 95° C. to 140° C. In some aspects, the flavor precursors mixture is heated for a time period of from 10 minutes to 60 minutes, from 60 minutes to 1200 minutes, from 120 minutes to 900 minutes, from 180 minutes to 720 minutes, from 240 minutes to 600 minutes, or from 300 minutes to 540 minutes. Preferably, the pressure for the Maillard reaction is from 0.1 to 10 bar, from 0.25 to 7.5 bar, from 0.4 to 5 bar, from 0.5 to 3 bar, from 0.75 to 2.5 bar, from 1 to 2 bar. Preferably the pH for the Maillard reaction is from 0.5 to 11, from 1 to 10.5, from 2 to 10, from 3 to 9, from 4 to 8.5, or from 5 to 8.

In some aspects, after the Maillard reaction the flavor composition obtained (MRP) can be dried on its own or mixed with a carrier like maltodextrin, through various methods known to the art, including spray drying, vacuum drying, freeze drying and such. In some aspects, other flavorants can be added to the MRP. In some aspects of the disclosure, the O. polymorpha-derived MRP comprises the addition of a Spiro-1,2,4-trithiolan compound is desirable, wherein Spiro compound is 3,5-bis(2′-Methyltetrahydrofuryl-3′) spiro-1,2,4-trithiolan. In some aspects, the amount of the Spiro-1,2,4-trithiolan compound is about 1% to about 50% of Spiro compound/100 g of MRP composition. In some aspects, the amount of the Spiro-1,2,4-trithiolan compound is about 5% to about 40%, about 10% to about 35%, about 15% to about 30%.

In some aspects, the O. polymorpha-derived MRP comprises the addition of other sources of MSG (e.g., purified from Corynebacterium glutamicum or other natural sources). In some aspects, the O. polymorpha-derived MRP comprises the addition of other sources IMP and/or GMP. In some aspects, the O. polymorpha-derived MRP comprises the addition of other sources of nutritious or flavoring peptides derived from mushroom extract and/or other natural sources.

Food products.

In some aspects, provided herein is a food product containing O. polymorpha cells, cell components or cell fractions.

In some aspects, provided herein is a food product containing an O. polymorpha extract.

In some aspects, provided herein is a food product containing a hydrolyzed O. polymorpha extract.

In some aspects, provided herein is a food product containing a flavor precursor mixture comprising O. polymorpha biomass, extract and/or hydrolyzed extract.

In some aspects, the O. polymorpha biomass, extract and/or hydrolyzed extract of the food product comprises at least 0.03% w/w, at least 0.05% w/w, at least 0.1% w/w, at least 0.15% w/w, at least 0.2% w/w, at least 0.225% w/w, at least 0.25% w/w, at least 0.275% w/w, at least 0.30% w/w, at least 0.4% w/w, at least 0.5% w/w, at least 0.6% w/w, at least 0.7% w/w, at least 0.8% w/w, at least 0.9% w/w, at least 1.0% w/w, at least 1.1% w/w, at least 1.2% w/w, at least 1.3% w/w, at least 1.4% w/w, at least 1.5% w/w, at least 1.6% w/w, at least 1.7% w/w, at least 1.8% w/w, at least 1.9% w/w, at least 2.0% w/w, at least 2.1% w/w, at least 2.2% w/w, at least 2.3% w/w, at least 2.4% w/w, at least 2.5% w/w, at least 2.6% w/w, at least 2.7% w/w, at least 2.8% w/w, at least 2.9% w/w, at least 3.0% w/w, or at least 3.1% w/w of heme.

In some aspects, the O. polymorpha biomass, extract and/or hydrolyzed extract of the food product comprises about 0.03% w/w, about 0.05% w/w, about 0.1% w/w, about 0.15% w/w, about 0.2% w/w, about 0.225% w/w, about 0.25% w/w, about 0.275% w/w, about 0.30% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, or about 3.1% w/w of heme.

In some aspects, the O. polymorpha biomass, extract and/or hydrolyzed extract of the food product comprises at least 0.8% w/w of glutamic acid. In some aspects, the O. polymorpha biomass, extract and/or hydrolyzed extract of the food product comprises at least 0.2% w/w of glutathione. In some aspects, the O. polymorpha biomass, extract and/or hydrolyzed extract of the food product comprises, at least, 0.007% of 5′ GMP. In some aspects, the O. polymorpha biomass, extract and/or hydrolyzed extract of the food product comprises at least 0.03% w/w of heme, 0.8% w/w of glutamic acid, 0.2% w/w of glutathione, and/or 0.007% of 5′ GMP.

In some aspects, the O. polymorpha biomass, extract and/or hydrolyzed extract of the food product comprises about 0.8% w/w of glutamic acid. In some aspects, the O. polymorpha biomass, extract and/or hydrolyzed extract of the food product comprises about 0.2% w/w of glutathione. In some aspects, the O. polymorpha biomass, extract and/or hydrolyzed extract of the food product comprises about 0.007% of 5′ GMP. In some aspects, the O. polymorpha biomass, extract and/or hydrolyzed extract of the food product comprises about 0.03% w/w of heme, about 0.8% w/w of glutamic acid, about 0.2% w/w of glutathione, and/or about 0.007% of 5′ GMP.

In some aspects, the disclosure provided herein discloses food products containing an O. polymorpha-derived MRP.

In some aspects of the invention, the food product is a solid or liquid (like a beverage). The food product of the invention can be a condiment, a baked good, a protein supplement, vitamin and mineral supplement, a meat product or a meat substitute product (meat analog). The meat product or the meat substitute can be a meat sausage replica, a meat pate replica, a grounded meat replica or a muscle replica. The condiment can be a sauce. The meat substitute can be free of animal directly derived products. The meat substitute may contain a small amount of animal directly derived products. An animal directly derived product is defined as an entire or part isolated from an animal, for example, isolated proteins, isolated tissue, isolated muscle, isolated cells. A recombinant protein produced in a non-animal host is not considered as a directly derived animal product in the context of this specification. A recombinant nucleic acid encoding for an animal protein is not considered as a directly derived animal product in the context of this specification.

A sauce can be a preparation, liquid or semiliquid, eaten as a relish accompanying food. The composition of the sauce of the invention can vary depending on the desired consistency.

Tofu is a traditional food product made from soy protein. The process generally consists of preparing soy milk by soaking and grinding soybeans, boiling the mixture, and filtering out remaining particulates. The soy kilf is further coagulated and pressed into solid blocks. In this way, Tofu can be considered as a plant-based protein-rich food.

Tempeh is an Indonesian-based protein-rich food manufactured from fermented soybean cake and cooked soybeans, e.g., rice and millet, shared with a Rhizopus oligosporus culture (Bakhsh, Allah, et al., “Traditional plant-based meat alternatives, current and a future perspective: A review” J. Agric. Life Sci 55:1-10 (2021)).

Seitan is a traditional food product made from wheat flour by activating gluten and washing out the starches. Alternatively, Seitan can be made from commercially obtained vital wheat gluten and the addition of water to hydrate. This may be followed by a heat treatment to cook the product. The resulting product has a chewy texture and can be added to a variety of dishes as an ingredient.

US Pub. No. 2005/003071 discloses plant-based meat analogues processed by sequentially blending methyl cellulose in ice/water mix, then blending in modified gluten and highly soluble vegetable protein in water and instigating gelling with mild heating, and oil to make an emulsion phase, and a modified starch. U.S. Pat. No. 7,070,827 discloses a process for making a vegetable protein meat analog involving sequentially blending methyl cellulose into a water/ice mix to form a cream, then blending in a modified gluten, a vegetable protein product having high solubility in water and capable of forming a gel with mild heat treatment, an oil to make an emulsion base, and a modified food starch and flavoring ingredients to form a flavored emulsion base. U.S. Pat. No. 10,039,306 discloses a meat replica matrix containing plant proteins, sugar, sulfur compound and a heme-containing protein. U.S. Pat. No. 10,863,761 discloses a meat replica containing a muscle replica, a fat tissue replica and a connective tissue replica. The muscle replica consists of a muscle replica formed by asymmetric fibers, like spun fibers or extruded fibers. The fat replica comprises a gel with droplets of fat suspended therein, wherein the fat can be vegetable oil, and the gel can be vegetable proteins. The connective tissue replica contains about 50% protein by total weight, about 50% by liquid weight, and has a low fat and polysaccharide component, wherein the protein is a prolamin.

In some aspects, the O. polymorpha derived compositions can be used as dietary supplements to complement the deficiencies of some minerals (like iron), amino acids, vitamins, and nucleotides in the human or animal diet.

A dietary supplement refers to a product intended for ingestion that, among other requirements, contains a “dietary ingredient” intended to supplement the diet. The dietary supplement containing O. polymorpha derived compositions may have many forms, such as pills, tablets, capsules, gummies, softgels, liquids, and powders.

In some aspects, the dietary supplement comprising O. polymorpha derived compositions can be used as a nutraceutical composition for treating anemia and other iron deficiencies related diseases. The dietary supplement may comprise dead cells of O. polymorpha where the cell may or may not be intact. The dietary supplement may comprise lysed cells of O. polymorpha. The dietary supplement may comprise a hydrolyzed extract of O. polymorpha. The dietary supplement may comprise a solution derived from the filtration of the hydrolyzed extract of O. polymorpha. The dietary supplement may comprise an insoluble fraction derived from the filtration of the hydrolyzed extract of O. polymorpha.

In some aspects, the O. polymorpha biomass, extract and/or hydrolyzed extract of the dietary supplement comprises at least 0.03% w/w, at least 0.05% w/w, at least 0.1% w/w, at least 0.15% w/w, at least 0.2% w/w, at least 0.225% w/w, at least 0.25% w/w, at least 0.275% w/w, at least 0.30% w/w, at least 0.4% w/w, at least 0.5% w/w, at least 0.6% w/w, at least 0.7% w/w, at least 0.8% w/w, at least 0.9% w/w, at least 1.0% w/w, at least 1.1% w/w, at least 1.2% w/w, at least 1.3% w/w, at least 1.4% w/w, at least 1.5% w/w, at least 1.6% w/w, at least 1.7% w/w, at least 1.8% w/w, at least 1.9% w/w, at least 2.0% w/w, at least 2.1% w/w, at least 2.2% w/w, at least 2.3% w/w, at least 2.4% w/w, at least 2.5% w/w, at least 2.6% w/w, at least 2.7% w/w, at least 2.8% w/w, at least 2.9% w/w, at least 3.0% w/w, or at least 3.1% w/w of heme.

In some aspects, the O. polymorpha biomass, extract and/or hydrolyzed extract of the dietary supplement comprises about 0.03% w/w, about 0.05% w/w, about 0.1% w/w, about 0.15% w/w, about 0.2% w/w, about 0.225% w/w, about 0.25% w/w, about 0.275% w/w, about 0.30% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, or about 3.1% w/w of heme.

In some aspects, the O. polymorpha derived compositions can be spread on the surface of the food. In some aspects, the O. polymorpha derived compositions can be solubilized or suspended and injected or mixed into food. In some aspects, spray dried O. polymorpha derived compositions can be mixed with the food.

### EXAMPLES

### Example 1. Media Optimization for Improved Growth and Heme Biosynthesis in O. polymorpha

1.1 Carbon and Nitrogen sources selection: To determine the cost effective and commercially feasible media for growth and heme biosynthesis using O. polymorpha yeast strain, one factor screening and optimizations were carried out using different carbon and nitrogen sources. These experiments were carried out in 500 mL Erlenmeyer shake flasks with a working volume of 100 mL in batch mode. The carbon sources tested were glucose, fructose, pure glycerol, and crude glycerol. The nitrogen sources evaluated were a synthetic media (SYN6), corn Steep liquor and corn protein hydrolysate. After the optimization, the media components, and the operational parameters were validated in 150 L bioreactors operated in fed-batch mode of fermentation.

1.2 Fermentation conditions: An YPD Agar plate (2% glucose) was inoculated with frozen cells from a glycerol cell banks culture and incubated for 45-65 hours at 30° C.-37° C. The seed-cultures were inoculated from a fresh YPD Agar plate into YPD Medium (2% w/v glucose) and grown overnight (20-25 h, 180 rpm at d=2.5 cm, 30° C.
37° C.).

In the bioreactor the cultivation was initiated in batch mode with carbon (glycerol) source concentration of 2% w/v, additional carbon (glycerol) source was continuously added in fed-batch mode over a period of 35-40 hours with a feed rate of 2-6 g/(1*h). The substrate feed, aeration, and the impeller speed (RPM) was adjusted in such a way that the dissolved oxygen is maintained between 20%-40% DO2 at maximum stirrer speed. During the fermentation the pH was set and maintained at 4.5-5.0 (corrective media: 12.5% (v/v) ammonia solution and 28% (v/v) phosphoric acid solution). Antifoam agent (10% (w/v) PEG 6000) was added as appropriate by the AF controller. Post cultivation for 40 hours, 0.5% v/v methanol was added as an inducing agent of oxidative stress and cultivation was further prolonged for additional 24 hours, prior to harvesting.

1.3 Cell lysis and extraction of heme: The cells were washed using sequential centrifugation and resuspension 3-4 times to remove insoluble or extracellular deposits or media components on the cell surface. After the final washing the cells were resuspended in a 20 mM NaOH PBS buffer for the cell disruption. For cell lysis disruption either Dyno mill, High-pressure homogenizer and/or ultrasonication were used. After the cell disruption the suspension was centrifuged to remove any insoluble particles and the supernatant can be stored at −20° C. for further use. The heme was extracted with 4 volumes of acidic acetone and incubated at −20° C. for 20 minutes, further centrifugation, and dilution of the sample 10× using acidic acetonitrile for quantification using HPLC.

1.4 Heme quantification: A HPLC method was developed using gradient flow using Acetonitrile (A) and 0.1% acetic acidic water (B) as mobile phase. The stationary phase: Luna 5 uM, C18 (2), 100 A, 250*4.6 mm column. Flow rate: 0.5 mL/min. Column pressure: 50-60 Kgf/cm2. Detector: SPD-M20A, Diode array detector. Wavelength: 406 nm. Hemin stock: Dissolve 41 mg of hemin (2.5 mM) in 25 mL of 20 mM NaOH in PBS. Hemin working stock: Dilute hemin stock 50 times (50 uM) using Acidic Acetonitrile (80% acetonitrile containing 20% 1.6M HCl), resulting in pink or red suspension (pH<5). HPLC standards: Using the 50 uM working stock, further samples were prepared by diluting to required concentrations using Acidic Acetonitrile.

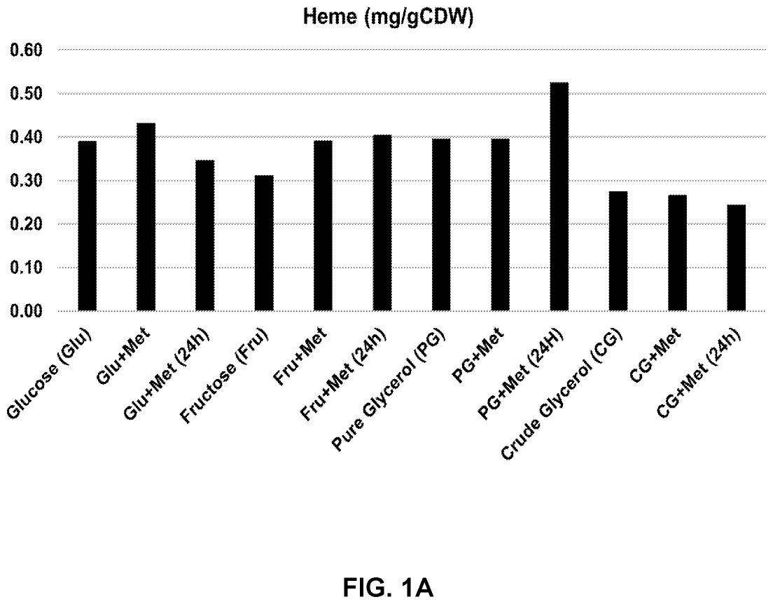

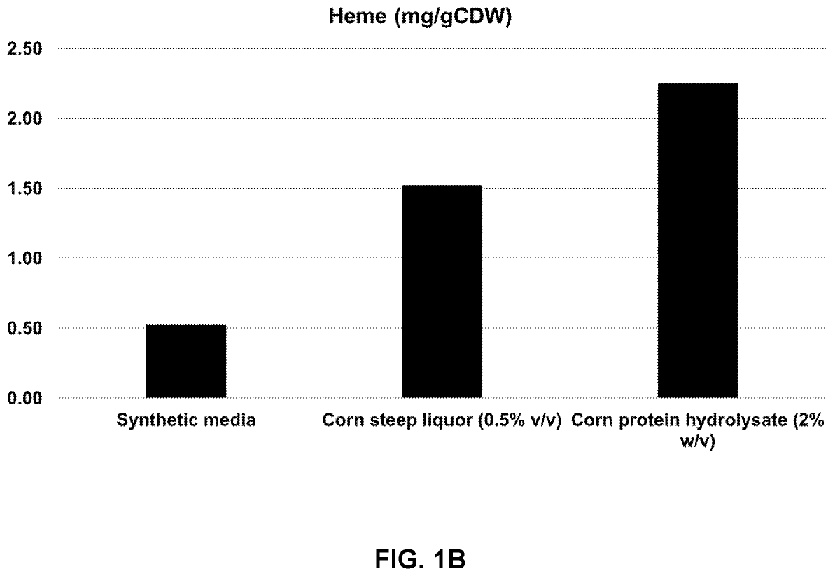

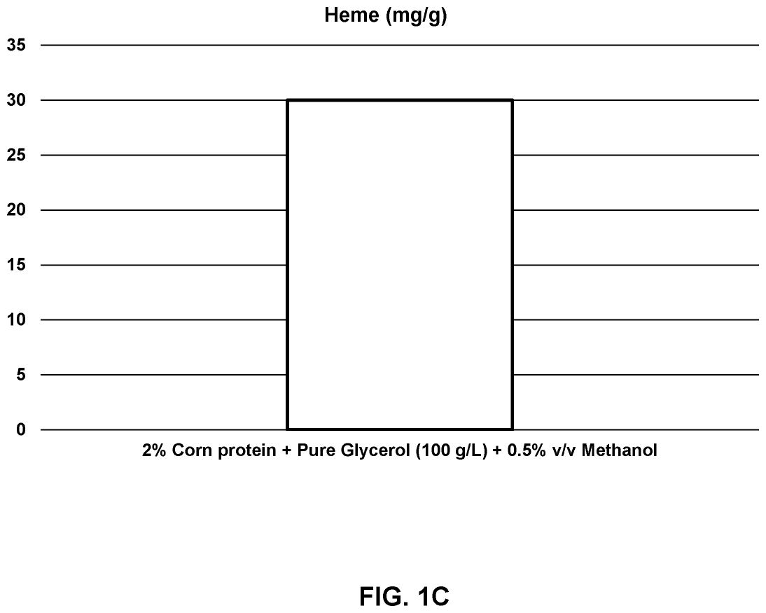

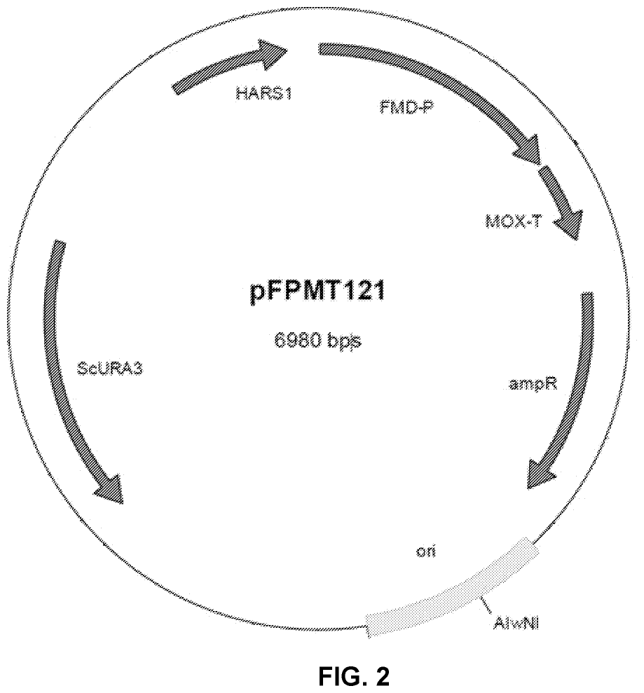

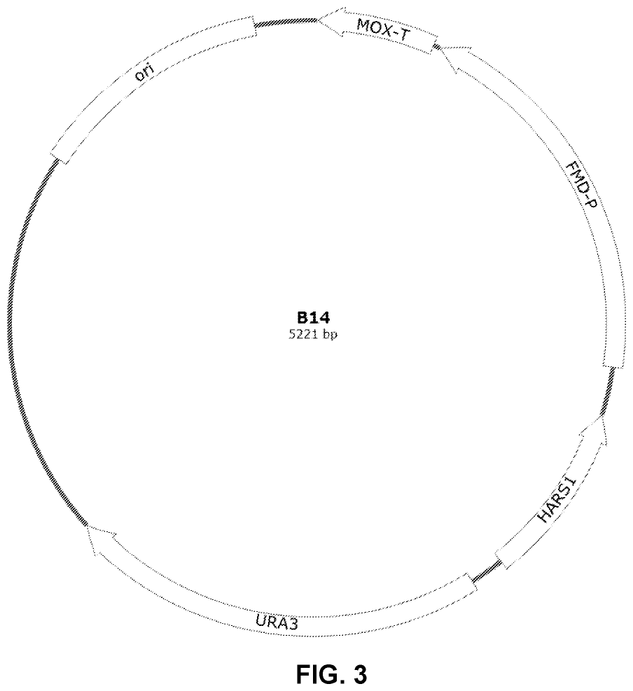

1.5 Results: FIG. 1A represents the yield of heme (per gram cell dry weight) obtained from O. polymorpha cultivated on different carbon sources with and without methanol induction. Glucose was the most preferred carbon source for the growth and development of any microorganism due to its reduced nature, resulting in highest cell dry weight (CDW) of 43.12 g/L with heme yield of 0.39 mg/g CDW (0.039% w/w), however the yield of heme with pure glycerol with methanol induction carried out after 24 hours was observed to have a highest yield of 0.53 mg/g CDW (0.053% w/w). Further experiments were carried out with glycerol as the preferred carbon source.

FIG. 1B depicts the heme production in relation to the nitrogen source. The heme yields increased 4 times (15 vs 60 mg/L) (0.053 vs 0.225% w/w) when the synthetic media (SYN6) was replaced with a complex medium containing of corn protein hydrolysate powder. With 2% w/v CPH powder, a heme yield of 2.25 mg per gram cell dry weight was observed.

FIG. 1C represents the heme quantification on the scale-up of the process at 150 L volumes, with optimal media parameters and process conditions observed in flask bench scale experiments, a maximum heme yield of 30 mg per gram cell dry weight (3% w/w) was observed.

1.6 Nutrition Profiling. Samples of two different batches were analyzed using standard ISO, AOAC, ELISA, spectrophotometry, gas chromatography (GC), high performance liquid chromatography (HPLC), and mass spectrophotometric (MS) techniques to evaluate the microbiological parameters, heavy metals, carbohydrates, total protein, dietary fiber, sugar profiling, vitamins, minerals, amino acids, carcinogens, aromatic volatiles etc. that could impact the nutritive value of the product described.

TABLE 1

| | Biomass | Biomass | O. polymorpha |
|---|---|---|---|
| Compound (% w/w) | (CPH) | (SYN6) | extract |
| Tryptophan (%) | 0.16 | 0.18 | 0.01 |
| Aspartic acid (%) | 0.5 | 0.07 | 2.33 |
| Serine (%) | 0.4 | 0.43 | 1.29 |
| Glutamic acid (%) | 0.86 | 0.95 | 4.04 |
| Glysine (%) | 0.24 | 0.31 | 1.62 |
| Histidine (%) | 1.65 | 1.36 | 0.41 |
| Arginine (%) | 0.21 | 0.33 | 0.18 |
| Threonine (%) | 0.26 | 0.27 | 0.95 |
| Alanine (%) | 0.45 | 0.35 | 2.31 |
| Proline (%) | 0.37 | 0.3 | 1.26 |
| Cysteine (%) | 0.09 | 0.15 | 0.01 |
| Tyrosine (%) | 0.23 | 0.29 | 0.43 |
| Valine (%) | 0.4 | 0.39 | 1.64 |
| Methionine (%) | 0.01 | 0.01 | 0.34 |
| Lysine (%) | 0.47 | 0.58 | 1.28 |
| Isoleucine (%) | 0.35 | 0.36 | 1.28 |
| Leucine (%) | 0.6 | 0.51 | 2.30 |
| Phenylalanine (%) | 0.23 | 0.22 | 1.32 |
| Total amino acids (%) | 7.48 | 7.06 | 22.98 |

Metals

| | | | |
|---|---|---|---|
| Iron (as Fe) (mg/kg) | 72.7 | 40.75 | 74.6 |
| Calcium (as Ca) (mg/kg) | 826.8 | 37.07 | 1085.8 |
| Phosphorous (as P) (mg/kg) | 4340.05 | 4594.22 | 11696.0 |
| Magnesium (as Mg) (mg/kg) | 359.12 | 532.1 | 2641.5 |
| Manganese (as Mn) (mg/kg) | 7.55 | ND | 20.5 |
| Sodium (as Na) (mg/kg) | 647.53 | 307.54 | 24334.6 |
| Potassium (as K) (mg/kg) | 3715.36 | 3999.83 | 12792.54 |
| Zinc (as Zn) (mg/kg) | 53.88 | 46.25 | 106.3 |
| Copper (as Cu) (mg/kg) | 10.35 | 5.23 | 4.3 |

### Example 2. Genetically Modified O. polymorpha Expressing a Recombinant Myoglobin

### 2.1-Myoglobin Genes Selection, Codon Optimization, and Synthesis.

Myoglobin (MG) gene from Bos taurus was codon-optimized (SEQ ID NO: 2) for expression in O. polymorpha and synthesized at ThermoFisher Scientific. Gene optimization not only increased the chances of efficient expression in the target host, but also facilitated cloning by removing undesired restriction sites.

### 2.2-Host Transformation and Selection

The O. polymorpha strain used was RB11 (ura3) and ALU3 (adel, leu2, ura3) and were obtained from Artes Biotechnology, GmbH.

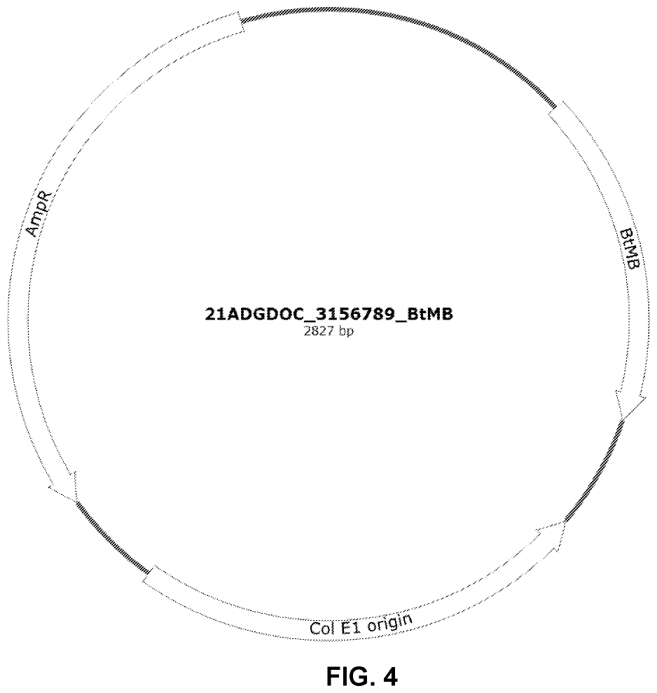

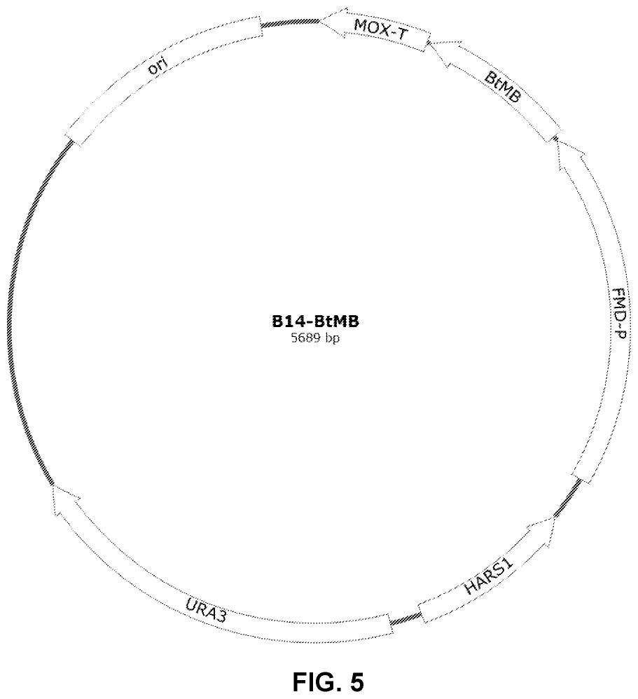

pFPMT121 and its derivatives were used as circular plasmids for the transformation of RB11 and ALU3 strains. B14 (FIG. 5), a derivative of pFPMT121, was the plasmid without the antibiotic resistance gene, which was used in this work for transformation of O. polymorpha RB11 strains.

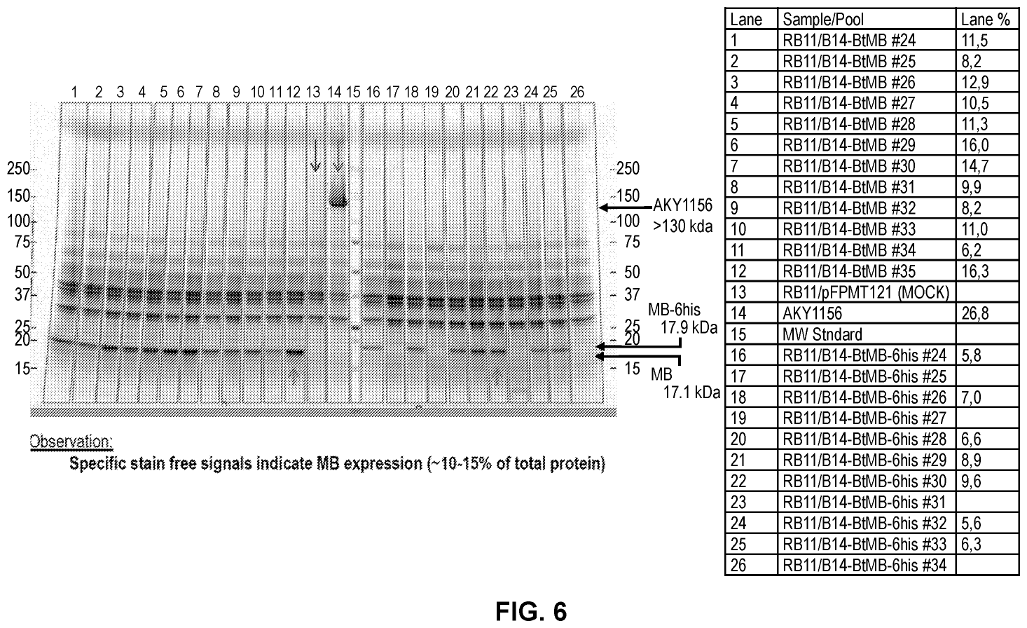

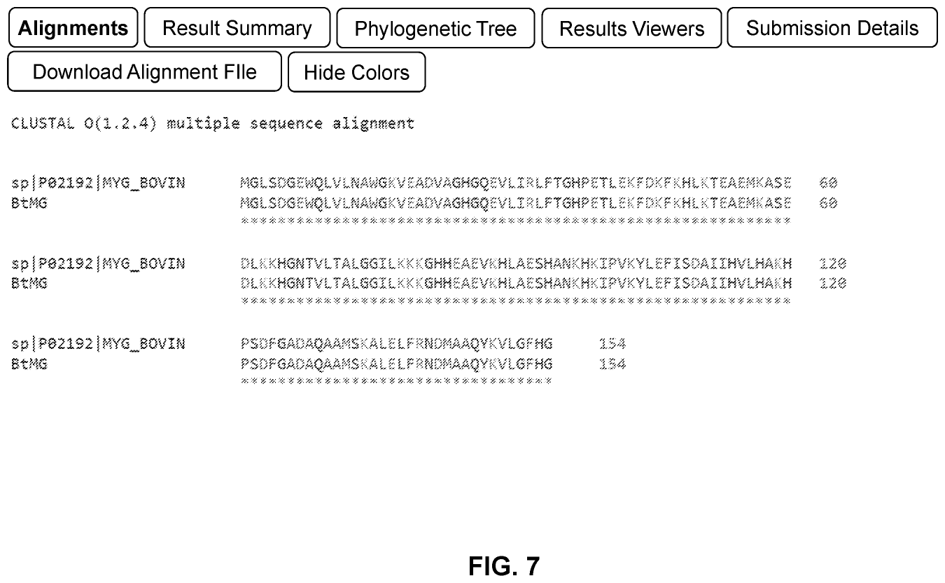

For construction of expression plasmid B14-BtMG (FIG. 6), the plasmid DNA supplied by Thermo Fisher Scientific/Gene art (FIG. 7) was cleaved with the restriction enzymes EcoRI and BamHI to yield a 480 bp gene (Myoglobin) fragment with optimized nucleotide sequences encoding for bovine myoglobin (BtMG). After electrophoresis of the restriction reaction, the fragment was purified from agarose gel. The vector plasmid B14 was also digested with EcoRI and BamHI overnight, and the linearized plasmid was gel-purified. Annealing the EcoRI overlaps creates a fusion of the heterologous gene to the promoter element (FMDp), joining the BamHI sites creates fusion of the heterologous gene to the terminator element (MOXt).

O. polymorpha RB11 and ALU3 strains were transformed based on the yeast electroporation protocols. 36 RB11 and 72 ALU3 transformants were screened using minimal (selective) and complex (non-selective) medium to achieve stable integration of the plasmids. Stain-Free imaging technology utilized a polyacrylamide gel containing a proprietary trihalo compound to make proteins fluorescent directly in the gel with a short photoactivation, which allowed the immediate visualization of proteins at any point during electrophoresis and western blotting. This trihalo compound was covalently bound to tryptophan residues, which enhanced their fluorescence when exposed to UV light, enabling the detection of proteins at levels as low as 10-25 ng.

The myoglobin expression levels were at least 10× higher in RB11 (10-16%) than ALU3 (3-6%) strains. A sample pool RB11/b14-BtMB #35 gave a very high expression of approximately 16.3% of total protein loaded onto the lane (FIG. 6). After the successful construction of the recombinant plasmid and transformation of O. polymorpha, sequencing was carried out of the plasmid and genome of the transformants. The polynucleotide sequence obtained are translated into open reading frames using Expasy Tool (ExPASy-Translate tool) and the acquired full length 3′-5′ ORF was compared to Bovine myoglobin sequence using CLUSTAL-multiple sequence alignment (MSA) tool. The sequence of the myoglobin gene integrated into the chromosome of O. polymorpha was observed to have 100% similarity with the bovine myoglobin gene (FIG. 7).

### 2.3-Production of Recombinant Myoglobin Protein in Bench-Top Bioreactors.

The production strain O. polymorpha RB11/b14-BtMB #35 was screened from 36 pools and cultivated in a 3L bioreactor for protein production and further characterization. The strain was cultivated in fed-batch mode of fermentation with an initial feed of 2% glycerol and de-repression was carried out by providing glycerol feed at 2-6 g/L per h until 48 h, as the culture attained stationary phase, the protein synthesis was induced by addition of 1% methanol and incubated for 24 hr.

The cell biomass was harvested and lysed using the bead mill to obtain the lysate containing myoglobin protein. The protein obtained was subjected to SDS PAGE and the quantification was performed through densitometric signals.

The sequence analysis confirmed the expression of 154 amino acid polypeptide chain similar to bovine myoglobin, and through densitometric analysis it was observed that O. polymorpha RB11/BtMG strain produced 1.6 g/L (˜ 10-15% of total protein) Myoglobin with a conversion yield of 20 mg/g cell dry weight. Further to that the information of heme loading is critical and trivial to determine the functionality of the protein, hence a heme quantification assay was optimized using heme assay kit (Heme Assay Kit sufficient for 250 colorimetric tests-Sigma-Aldrich (sigmaaldrich.com)). The colorimetric assay was carried out using commercial myoglobin (Left) (animal origin) and Moolec myoglobin (Right) resulting in 200 uM (8.6 mg/mL) and 30 uM (1.6 mg/mL), respectively.

### 2.4-Production of Recombinant Myoglobin Protein in Semi-Pilot Scale (10 L) Bioreactors.

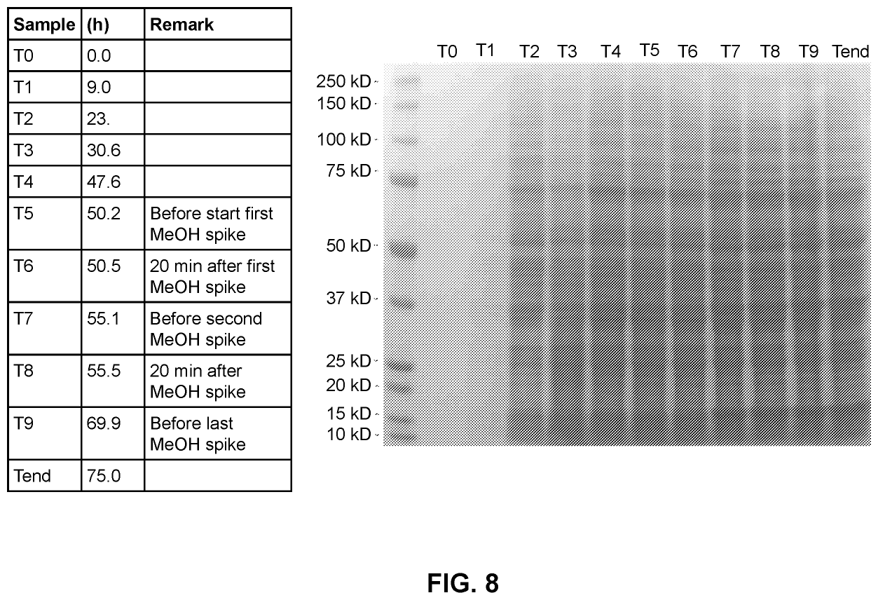

The production was initiated in a Sartorius C-DCU 15-L stainless steel fermenter (C-DCU 15-L) as a batch with 2% glycerol as the sole carbon source; further fed-batch mode of fermentation was initiated at 12 h when the concentration of glycerol in the production media was less than 10 g/L. The linear feeding of glycerol was carried out from 12 h to 50 h of cultivation with a feed rate of 2-6 g/L per h. The substrate feed rate and the dissolved oxygen (DO) concentration in the reactor was adjusted, such a way 20-40% DO measurement was maintained throughout the incubation time at maximum stirrer speed. During the fermentation the pH was set and maintained at 4.8 (corrective media: 20% (v/v) ammonia solution and 20% (v/v) phosphoric acid solution). Antifoam agent (10% (w/v) Structol J 673) was added as appropriate by the AF controller. After 50 h of cultivation the methanol induction was executed by batch-wise addition of methanol according to the following scheme: 0 h and 5 h: addition of each 1.0% (v/v) methanol-sol; 8 h, 11 h, 14 h and 17 h: addition of each 0.5% (v/v) methanol-sol (FIG. 8).

2.5-Separation and Purification of Myoglobin from the Accumulated Cell Biomass

The microbial cell biomass generated was harvested through bucket centrifuge, cell pellet was resuspended in soft water and further spin down. The process was repeated for at least 3 times, at each step a sample of known volume was collected for qualitative and quantitative characterization of microbial cells, metabolites, and other characteristic compounds. After the final washing step, the cells were resuspended in a lysis buffer for the cell disruption.

The microbial cells were lysed by passing the suspension from step 1 through Dyno mill in 3 passes. During the milling, the suspension was warmed up to 30° C. After cell disruption, the suspension was centrifuged for 60 min at 6362 RCF. Thereafter the supernatant was collected and frozen at −20° C.

In the current disclosure, the myoglobin expressed was intracellular, hence the microbial biomass accumulated post cultivation was processed through following steps for the separation and purification.

### 2.5.1-Cell Wash:

The microbial cell biomass generated through fed-batch cultivation was harvested through bucket centrifuge (630 RS), cell pellet was resuspended in soft water and further spin down at 6362 RCF. The process was repeated for at least 3 times, at each step a sample of known volume was collected for qualitative and quantitative characterization of microbial cells, metabolites, and other characteristic compounds. After the final washing step, the cells were resuspended in a lysis buffer for the cell disruption.

### 2.5.2-Quantification of Microbial Growth:

5 mL of each sample was weighed and filled with about 45 mL deionised water. The suspension was centrifuged for 10 min at 3333 g and a second round of 5 min at 5000 g. The pellet was collectively transferred to a pre weighed glass vial. Then, it was incubated at 93° C. overnight and in the morning for 5 h at 105° C. Finally, the dry weight is calculated in g/kg.

### 2.5.3-Cell Disruption:

The microbial cells were lysed by passing the suspension from step 1 through Dyno mill in 3 passes. During the milling, the suspension was warmed up to 30° C. After cell disruption, the suspension was centrifuged for 60 min at 6362 RCF. Thereafter the supernatant was collected and frozen at −20° C.

### 2.5.4-Protein Recovery and Purification:

The cell lysate obtained from 2.9—was subjected to micro and ultrafiltration passing through different cutoff membranes with the sizes in the range of 5 kDa to 800 kDa.

### 2.5.5-Qualitative Myoglobin Analysis:

The samples collected during the methanol induction and downstream process were qualitatively analyzed for myoglobin using SDS PAGE. The sequence analysis confirmed the expression of 154 amino acid polypeptide chain similar to bovine myoglobin, and through densitometric analysis it was observed that O. polymorpha RB11/BtMG strain produced 1.6 g/L (˜ 10-15% of total protein) myoglobin with a conversion yield of 20 mg/g cell dry weight.

The protein band pattern in the SDS PAGE analysis (FIG. 8) shows the appearance of a characteristic 17 kDa band post methanol induction (T7 lane), maintaining its appearance until the end of the induction (T End Lane). Similarly, the samples obtained at the end of each step in the downstream process were qualitatively analyzed for myoglobin protein.

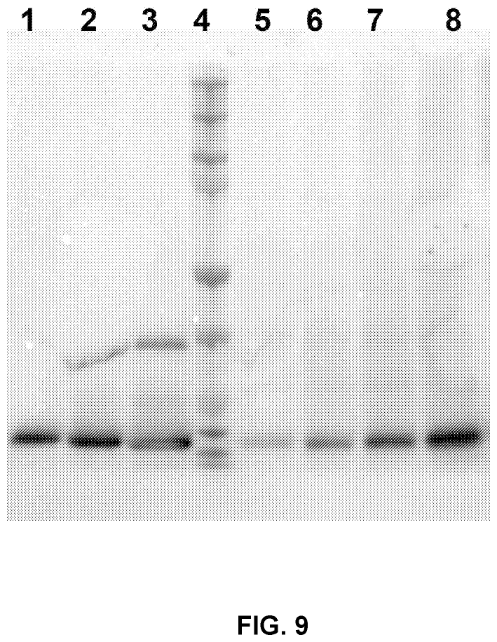

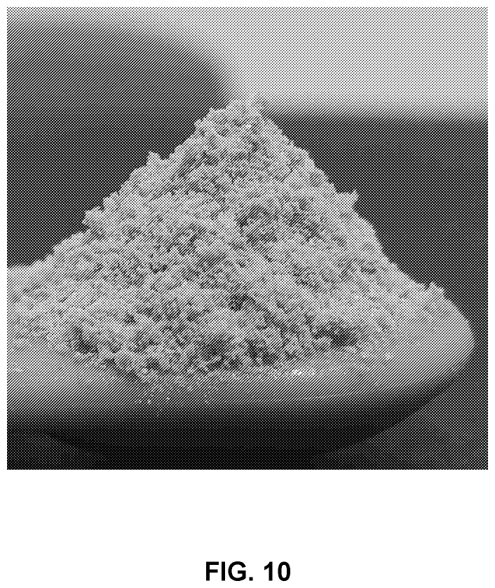

From the western blot analysis (FIG. 9), the myoglobin characteristic band around 17 kDa was observed from the final sample.

### 2.5.6-Quantification of Heme:

Further to that the information on quantification of recombinant myoglobin produced by engineered O. polymorpha strains, heme loading was vital to determine the functionality of the protein, hence a heme quantification assay was optimized using a heme assay kit (Heme Assay Kit sufficient for 250 colorimetric tests Sigma-Aldrich (sigmaaldrich.com)).

The procedure for the heme assay is as follows: As per the manufacturer protocol, 50 μl of the purified myoglobin is mixed with the 200 μl of the reagent, and incubated for 5 minutes at room temperature. After the incubation, sample absorbance was recorded at 400 nm using a spectrophotometer. Simultaneously a linear standard graph can be prepared using heme calibrator by making different dilutions. The protocol said, the provided heme calibrator corresponds to 62.5 uM heme, with a linear detection range of 0.6-125 uM.

The optic density read was 0.148 corresponding to 300.95 uM of heme, which corresponds to 501.58 uM myoglobin with 60% heme loading.

TABLE 2

| | | | | % heme loading |
|---|---|---|---|---|
| | | | Heme | (μMₕₑₘₑ/ |
| | | Myoglobin | quantified | μM_{Myoglobin} * |
| | Sample | (μM) | (μM) | 100) |
| 1 | Homogenized yeast cells in | 7.48 | 4.49 | 59.89 |
| | 50 mM Phosphate buffer | | | |
| 2 | 5 kDa retenate in 50 mM | 65.78 | 39.47 | 60.00 |
| | phosphate buffer | | | |
| 3 | 5 kDa retenate | 501.58 | 300.95 | 60.00 |
| | (10x concentrated) | | | |
| 4 | 1.4 mg/mL Myoglobin + | 152.35 | 91.41 | 60.00 |
| | 62.5 μM Heme (Control) | | | |
| 5 | 0.5 mg/mL Myoglobin + | 116.71 | 70.02 | 59.99 |
| | 62.5 μM Heme (Control) | | | |

Further the sample obtained from 5 kDa filter module was quantified for myoglobin concentration and the heme loading, which resulted in 30-40 g/L myoglobin with 60%-70% purity, and 50%-60% heme loaded on to apomyoglobin.

2.6-Production of Recombinant Myoglobin in Pichia pastoris:

To provide further details that support the efficiency of O. polymorpha as the suitable host for the production of heme-containing proteins, the myoglobin (MG) gene from Bos taurus was codon-optimized for expression in Pichia pastoris. The myoglobin gene was expressed under the AOX1 promoter and terminator sequences.

The positive transformants obtained after the myoglobin gene transformation and confirmation, were cultivated in yeast extract, peptone, and dextrose (YPD) medium for expression studies.

The single colony of the positive transformant from the plate screened by PCR identification was inoculated into 10 ml YPD liquid culture.

After 24 h, the culture medium was milky white, and 10 ml of culture medium was inoculated into 1L YPD medium (inoculation amount 1%), and incubated at 28° C., 230 rpm.

Preparation with methanol for induction: hemin was dissolved with 0.2 M NaOH, the concentration of hemin was 100 mM, then diluted 10 times with methanol, and sterile filter to obtain methanol containing 10 mM hemin.

When the OD600 value of bacteria reached 0.6-0.7 after 50 times dilution (about 24 h), the culture solution was poured into two sterilized and cooled 500 ml Tubes were centrifuged on the ultra-clean workbench and centrifuged at 6000 rpm, 4° C., for 5 min. The supernatant was poured out, the yeast resuspended with 900 ml YP medium, and the resuspended system poured back into a 5 L conical flask. Sterilized 100 ml 1 M phosphate buffer was added (the working concentration of phosphate is 0.1 M), and 10 ml methanol containing 10 mM hemin was added to the system to start induction (the working concentration of methanol is 1%, the working concentration of hemin is 0.1 mM).

10 ml of methanol containing 10 mM hemin was added to the system every 24 h.

Samples were taken every day for TCA concentrated electrophoresis. The time when methanol is added was noted for the first time as zero time. After about 48 hours (different proteins may have different induction time), the culture solution was poured into two 500 ml Centrifuge Tubes, 6000 rpm, and centrifuged at 4 for 10 minutes. Supernatant was collected and frozen at −20° C. for further use.

As explained above, without the addition of external hemin, no heme loading onto the myoglobin protein was observed; however, after the supplementation of external heme, myoglobin loaded with heme up to 22%.

### Example 3-O. Polymorpha Cell Lysis

O. polymorpha frozen biomass was thawed to room temperature and thereafter diluted in tapwater to a concentration of approximately 30% dry matter (moisture analyzer, Mettler Toledo HB43). Two conditions were made, control biomass (pH 5.2) and biomass adjusted to pH 8.5 (addition of 33% NaOH) in combination with 2.5% Alcalase enzyme (Novozymes, 2.4 L pure (2.4 AU-A/g). Both biomass solutions were incubated at 50° C. for 4 hours with continuous shaking. The pH of the Alcalase sample was kept at approximately 8.5 by the addition of small 33% NaOH volumes during the process. Samples were collected every hour and stored at −10° C. for subsequent analysis.

### Example 4. Enzymatic Hydrolysis

After 4 hours of incubation, the control biomass sample was finished. The Alcalase treated biomass was first set at pH 5.5 using 5% hydrochloric acid, after which the first enzymes were added (Step 1), RP-1G (0.16%, Amano Enzyme, a 5′ phosphodiesterase that hydrolyses RNA to 5′-nucleotides) and Deamizyme (0.067%, Amano Enzyme, converts the nucleotide 5′ AMP to 5′ IMP). The nucleotides 5′ GMP and 5′ IMP are known flavor enhancers. The enzymes were incubated for 5 hours at 50° C. while shaking.

Next, the solution was set at pH 7.0 with 33% NaOH, after which the second enzyme mix was added (Step 2). Protana UBoost (0.17%, Novozymes, glutaminase that generates glutamate from glutamine), Protana Prime (0.17%, Novozymes, exo-peptidase mix able to release free amino acids) and ProteAXH (0.17%, Amano Enzyme, exhibits protease and peptidase activities) are known to contribute to umami flavor enhancement and were incubated for 12 hours at 50° C. while shaking. Samples were collected after every enzymatic digestion step.

In order to determine that the process of lysis and hydrolysis is not degrading the heme, a sample was of each step was taken and heme concentration was determined according to the methodology used in example 1 (1.4), as shown in Table 3.

TABLE 3

| Sample | Heme (% w/w) |
|---|---|
| Crude biomass | 2.9 |
| Cell lysate | 2.2 |
| Step 1 | 3.1 |
| Step 2 | 2.4 |

### Example 5-Micro—and Ultrafiltration

The hydrolyzed product was first separated by microfiltration. A 800 kDa membrane was installed in the filtration system of SANI Membranes (Vibro-Lab3500), after which the digested solution was pumped through the membrane module in combination with continuous vibration. The retentate (insoluble fraction) consisted of large polysaccharides (beta-glucans, a group of glucose polymers that form the yeast cell wall) and the permeate represented the intercellular parts of the yeast cell (clear, dark brown solution). Subsequently, the 800 kDa permeate (soluble fraction) was used for ultrafiltration using a 1 kDa membrane which generated a product of soluble small peptides and nucleotides collected in the permeate.

### Example 6-Quantification of GMP

The spray dried fraction of yeast hydrolysate obtained after the filtration (obtained after the examples 5) was utilized for determining the concentrations of flavour enhancing GMP using isocratic or gradient HPLC elution method using 0.5% orthophosphoric acid (A) and 100% methanol (B) as mobile phase, and Gemini Phenyl C6 reverse phased 5 μm, 110 A, 50*4.6 mm column as stationary phase. The flow rate was maintained at 0.5 mL/min, with column pressure: 50-60 Kgf/cm2. Detector: SPD-M20A, Diode array detector. Wavelength: 254 nm.

Results: An average concentration of 0.007% w/w of GMP was observed per gram of O. polymorpha yeast hydrolysate.

### Example 7-Quantification of Glutathione

The yeast hydrolysate obtained after the filtration was utilized to understand the concentrations of glutathione, the UMAMI flavour ingredient using UPLC-QTOF through C18 column, subjected to positive electron spray ionization. The gradient elution method comprised 0.1% formic acid water (A) and 0.1% formic acid in Acetonitrile (B) as mobile phase, and Acquity UPLC BEH C18, 50 * 2.1 mm column as stationary phase. The flow rate was maintained at 0.25 mL/min, with MS scan range of 100-2000 m/z.

Results: An average concentration of 11.62% w/w of glutathione oxidized and 0.29% w/w of glutathione reduced was observed per gram of spray dried yeast product.

### Example 8-Flavorant Derived from O. polymorpha

8.1-Flavorant 1. O. polymorpha yeast extract (20%-24% ds) as protein source (370 g), amino acids (cysteine, methionine, glutamine, glycine) 16 g, Vitamin B1 (3-5 g), a reducing carbohydrate source (d-Xylose, Ribose, Arabinose, Rhamnose, glucose) 40 g, water 80 g, caustic 33% (8-10 g), were mixed together (Mixture 1) with a high shear mixer and poured into a Parr high pressure stirred reactor. The slurry was heated to 120° C. within 30 min. After reaching 120° C. the slurry was kept at this temperature for 60 minutes (pressure build up was observed: 3 bar). After the reaction the batch was cooled down to 50° C. within 30 minutes. The resulting liquid was blended with an amount of carrier and was subsequently spray dried or vacuum dried or left as a liquid.

8.2-Flavorant 2. O. polymorpha extract 20%-24% ds (200 g), amino acids (Lysine, Arginine, Histidine, Cysteine 25 g), taste enhancer IMP/GMP 5 g, acids (lactic, succinic, tartaric) 15 g, a reducing carbohydrate source (glucose, arabinose) 120 g, caustic 33% 37 g were mixed together (Mixture 2) with a high shear mixer and poured into a Parr high pressure stirred reactor. The slurry was heated to 120° C. within 30 minutes. After reaching 120° C. the slurry was kept at this temperature for 30 minutes. After the reaction the batch was cooled down to 50° C. within 30 minutes. The resulting liquid was used as such or blended with an amount of carrier and was subsequently spray dried or vacuum dried.

8.3 Flavorant 3. A cell culture of a genetically modified O. polymorpha expressing myoglobin was lysed with Papain (0.1%) for 2 hours at 65° C., followed by 24 hours with 0.05% Protease A Amano 2SD (Amano Enzyme Inc.) at 50° C. 580 g of the genetically modified O. polymorpha extract was mixed with 15.8 g of a yeast extract, 60 g of gum acacia, 120 g of water, 14 g of amino acids (Gly, Ala, Cys and Met), 0.35 g of Vitamin B1, 3.6 g of reducing sugars (Rhamnose, Arabinose, ribose, D-xylose) and 7.2 g of disodium phosphate as a buffer (Mixture 3). The mixture was thermally treated at 120° C. during 30 minutes at a pressure between 1-2.5 bar at pH=6.5, to produce a Maillard reaction product (Flavorant 3).

8.4 Flavorant 4. The Maillard Reaction Product of flavorant 3 (750 g) was mixed with maltodextrin (255 g), gum acacia (45 g) and water (380 g) and spray dried. The spray dried mixture (280 g) was further mixed with other ingredients: 75 g of Furaneol, 90 g of yeast extract KU012, 100 g of yeast extract KA65, 160 g of a mixture of organic acids (lactic, malic, citric, tartaric, succinic and acetic acids) and amino acids (glycine, alanine, cysteine, glutamine, methionine) and salt (200 g). The resultant flavorant was tasted at 0.4% plus 0.2% of salt in lukewarm water giving a distinct meaty flavor with metallic bloody aftertaste.

### Example 9-Application to Food Products

9.1-Sauce. Soy sauce 10%, Flavorant 1 5%, salt 19%, water 56%, beef flavor topnote 0.5%, spice extracts (paprika, chilli, garlic, onion) 0.1%, taste enhancers IMP/GMP/msg (msg is monosodium glutamate) 3.5%, tomato paste 1%, molasses 3%, Flavorant 2 1%, xanthan gum 0.3%. The product was mixed and heated to 80° C. and hot packed in a container. The product had a nice round korean style beef taste.

9.2-Food matrices. Several food matrices have been developed based on well known techniques. The incorporation of O. polymorpha cells, extracts, hydrolyzed extract and flavorants were incorporated into these food matrices. The resume of the food products and the main ingredients is shown in Tables 4-6.

TABLE 4

| | Product type | | | | |
|---|---|---|---|---|---|
| | | | | | Whole |
| | Emulsified | Coarse | | | muscle-type |
| | products | products | | | products |
| | Technique | | | | |
| | | | | | High moisture |
| | | Reconstituted | Reconstituted | Hydrated | meat |
| | Emulsion | products | products | TVP | analogues |
| | Breaded | | | | |
| | | breaded | non-breaded | | |
| | Examples | | | | |
| | | | burgers, | | |
| | Sausages, | nuggets, | meatballs, | Minced | Chicken |
| | frankfurters, | schnitzel, | chicken breast, | meat, | strips, steak- |
| | bologna | fish finger | salmon | shoarma | like cuts |
| Water | 50 to 80 | 40 to 60 | 50 to 80 | 50 to 70 | 50 to 75 |
| Textured | 10 to 40 | 10 to 30 | 10 to 40 | 20 to 30 | 10 to 25 |
| vegetable | | | | | |
| proteins | | | | | |
| Non-textured | 4 to 20 | 4 to 10 | 4 to 20 | n/a | n/a |
| proteins | | | | | |
| Flavors/spices | 3 to 10 | 3 to 5 | 3 to 10 | 2 to 5 | 2 to 5 |
| O. polymorpha | 0.1 to 10 | 0.1 to 10 | 0.1 to 10 | 0.1 to 10 | 0.1 to 10 |
| Fat/oil | 10 to 15 | 5 to 10 | 5 to 15 | 0.5 to 10 | 0.5 to 5 |
| Binding agents | 1 to 5 | 15 to 20 | 1 to 5 | n/a | n/a |
| Coloring agents | 0 to 1 | 0 to 1 | 0 to 1 | 0 to 0.5 | 0 to 0.5 |

*binding agents: wheat gluten, egg whites, and hydrocolloids enzymes, starches

TABLE 5

| | | | Chicken |
|---|---|---|---|
| | Food | | Nuggets |
| Component | Product | Burger | (breaded) |

| | | |
|---|---|---|
| Water | 58 | 50 |
| Textured vegetable proteins | 14 | 15 |
| Non-textured proteins | 3 | 2 |
| Flavors/spices | 6 | 5 |
| Fat/oil | 15 | 10 |
| Binding agents:* | 3 | 17 |
| O. polymorpha | 0.1-10 | 0.1-10 |
| Coloring agents | 1 | 1 |
| Total | 100 | 100 |

*Such as: wheat gluten, egg whites, and hydrocolloids enzymes, starches

TABLE 6

| Flavors/spices in | | |
|---|---|---|
| chicken nugget (breaded) | Breading | Patty |
| Breading and Patty % of total | 45% | 55% |
| product weight | | |
| O. polymorpha derived | 6% of | 4.2% of |
| compositions in breading or patty | breading | patty |
| Amount of flavoring and spices in | 2.7% of final | 2.31% of |
| the final product in the breading or | product | final product |
| patty fraction | | |

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

TABLE 7
Sequences

| SEQ | | |
|---|---|---|
| ID | | |
| NO: | Description | Sequence |
| 1 | >BC102986.1 | AAAAACCCCAGCTGTCGGAG |
| | Bos taurus | ACAGGACACCCAGTCAGTCC |
| | myoglobin, mRNA | GCCCTTGTTCTTTTTCTCTT |
| | (cDNA clone | CTTCAGACTGCGCCATGGGG |
| | MGC: 128380 | CTCAGCGACTGGGAATGGCA |
| | IMAGE: 7986409), | GTTGGTGCTGAATGCCTGGG |
| | complete cds | GGAAGGTGGAGGCTGATGTC |
| | | GCAGGCCATGGGCAGGAGGT |
| | | CCTCATCAGGCTCTTCACAG |
| | | GTCATCCCGAGACCCTGGAG |
| | | AAATTTGACAAGTTCAAGCA |
| | | CCTGAAGACAGAGGCTGAGA |
| | | TGAAGGCCTCCGAGGACCTG |
| | | AAGAAGCATGGCAACACGGT |
| | | GCTCACGGCCCTGGGGGGTA |
| | | TCCTGAAGAAAAAGGGTCAC |
| | | CATGAGGCAGAGGTGAAGCA |
| | | CCTGGCCGAGTCACATGCCA |
| | | ACAAGCACAAGATCCCTGTC |
| | | AAGTACCTGGAGTTCATCTC |
| | | GGACGCCATCATCCATGTTC |
| | | TACATGCCAAGCATCCTTCA |
| | | GACTTCGGTGCTGATGCCCA |
| | | GGCTGCCATGAGCAAGGCCC |
| | | TGGAACTGTTCCGGAATGAC |
| | | ATGGCTGCCCAGTACAAGGT |
| | | GCTGGGCTTCCATGGCTAAG |
| | | CCCCACCCCTGTGCCCCTCA |
| | | CCCCACCCACCTGGGCAGGG |
| | | TGGGCGGGGACTGAATCCCA |
| | | AGTAGTTATAGGGTTTGCTT |
| | | CTGAGTGTGTGCTTTGTTTA |
| | | GGAGAGGTGGGTGGAAGAGG |
| | | TGGATGGGTTAGGGGTGGAG |
| | | GGAGCCTTGGGAGAGGCCTG |
| | | GGGACCAGGCTTTCAGTGGA |
| | | GGGTCATCAACTTGGGAACC |
| | | ATGAGAAGCTTGACTGTGGC |
| | | TGGCTGAGTCTGGGTCAAAC |
| | | TCAACTTTCCTTTCACCTCA |
| | | ATGCCAACCCAATTCCTACC |
| | | AACCTCTAAACTGACCTGCA |
| | | CCTTTACCCTCACCTTAAAT |
| | | CCCCAATCCGAGCTGTCAAC |
| | | ATAAACTCCAGCCTAATTCT |
| | | CTGACCCCATCACCCAGCCC |
| | | CTTGAAGACAGCAGAGTGTC |
| | | TTGCTTGCCCTGAGAAGGAA |
| | | GTGTGGGCCGGGTGGGACGG |
| | | CCACACCCAGCCCTAGGGAG |
| | | GCATGGAGGCATGGTGTCTG |
| | | CAACATAAATGTCCCTTCTC |
| | | AGGTAGGGGAGTGACACCTG |
| | | GTTTAATAAAGGATTTCTCA |
| | | CATCACAAAAAAAAAAAAAA |
| | | AA |
| 2 | Myoglobin cDNA | GTTTCAATTCATCATTTTTT |
| | codon-optimized | TTTTATTCTTTTTTTTGATT |
| | sequence | TCGGTTTCCTTGAAATTTTT |
| | | TTGATTCGGTAATCTCCGAA |
| | | CAGAAGGAAGAACGAAGGAA |
| | | GGAGCACAGACTTAGATTGG |
| | | TATATATACGCATATGTAGT |
| | | GTTGAAGAAACATGAAATTG |
| | | CCCAGTATTCTTAACCCAAC |
| | | TGCACAGAACAAAAACCTGC |
| | | AGGAAACGAAGATAAATCAT |
| | | GTCGAAAGCTACATATAAGG |
| | | AACGTGCTGCTACTCATCCT |
| | | AGTCCTGTTGCTGCCAAGCT |
| | | ATTTAATATCATGCACGAAA |
| | | AGCAAACAAACTTGTGTGCT |
| | | TCATTGGATGTTCGTACCAC |
| | | CAAGGAATTACTGGAGTTAG |
| | | TTGAAGCATTAGGTCCCAAA |
| | | ATTTGTTTACTAAAAACACA |
| | | TGTGGATATCTTGACTGATT |
| | | TTTCCATGGAGGGCACAGTT |
| | | AAGCCGCTAAAGGCATTATC |
| | | CGCCAAGTACAATTTTTTAC |
| | | TCTTCGAAGACAGAAAATTT |
| | | GCTGACATTGGTAATACAGT |
| | | CAAATTGCAGTACTCTGCGG |
| | | GTGTATACAGAATAGCAGAA |
| | | TGGGCAGACATTACGAATGC |
| | | ACACGGTGTGGTGGGCCCAG |
| | | GTATTGTTAGCGGTTTGAAG |
| | | CAGGCGGCAGAAGAAGTAAC |
| | | AAAGGAACCTAGAGGCCTTT |
| | | TGATGTTAGCAGAATTGTCA |
| | | TGCAAGGGCTCCCTATCTAC |
| | | TGGAGAATATACTAAGGGTA |
| | | CTGTTGACATTGCGAAGAGC |
| | | GACAAAGATTTTGTTATCGG |
| | | CTTTATTGCTCAAAGAGACA |
| | | TGGGTGGAAGAGATGAAGGT |
| | | TACGATTGGTTGATTATGAC |
| | | ACCCGGTGTGGGTTTAGATG |
| | | ACA |
| 3 | FMD promoter | AGGGAGACGCATTGGGTCAA |
| | sequence | CAGTATAGAACCGTGGATGA |
| | | TGTGGTCTCTACAGGATCTG |
| | | ACATTATTATTGTTGGAAGA |
| | | GGACTATTTGCAAAGGGAAG |
| | | GGATGCTAAGGTAGAGGGTG |
| | | AACGTTACAGAAAAGCAGGC |
| | | TGGGAAGCATATTTGAGAAG |
| | | ATGCGGCCAGCAAAACTAAA |
| | | AAACTGTATTATAAGTAAAT |
| | | GCATGTATACTAAACTCACA |
| | | AATTAGAGCTTCAATTTAAT |
| | | TATATCAGTTATTACCCGGG |
| | | AATCTCGGTCGTAATGATTT |
| | | TTATAATGACGAAAAAAAAA |
| | | AATTGGAAAGAAAACCGATG |
| | | AAGGCAGAGAGCGCAAGGAG |
| | | GCGGTATTTATAGTGCCATT |
| | | CCCCTCTCTGAGAGACCCGG |
| | | ATGGTAGTCGAGTGTATCGG |
| | | AGACAGCTTGATGTAGACTC |
| | | CGTGCCTGCCGGCTCCTCTT |
| | | ATTGGCGGACACCAGTGAGA |
| | | CACCCCGGAACTTGCTGTTT |
| | | TTCTGCAAAATCCGGGGTGA |
| | | CCAGTGGGAGCCTATTTGCA |
| | | CACACGAGCGGGACACCCCA |
| | | CTCTGGTGAAGAGTGCCAAA |
| | | GTCATTCTTTTTCCCGTTGC |
| | | GGGGCAGCCGATTGCATGTT |
| | | TTAGGAAAATATTACCTTTG |
| | | CTACACCCTGTCAGATTTAC |
| | | CCTCCACACATATATATTCC |
| | | GTCACCTCCAGGGACTATTA |
| | | TTCGTCGTTGCGCCGCCAGC |
| | | GGAAGATATCCAGAAGCTGT |
| | | TTTCCGAGAGACTCGGTTGG |
| | | CGCCTGGTATATTTGATGGA |
| | | TGTCGCGCTGCCTCACGTCC |
| | | CGGTACCCAGGAACGCGGTG |
| | | GGATCTCGGGCCCATCGAAG |
| | | ACTGTGCTCCAGACTGCTCG |
| | | CCCAGCAGGTGTTTCTTGAT |
| | | CGCCGCCTCTAAATTGTCCG |
| | | CGCATCGCCGGTAACATTTT |
| | | TCCAGCTCGGAGTTTGCGTT |
| | | TAGATACAGTTTCTGCGATG |
| | | CCAAAGGAGCCTGCAGATTA |
| | | TAACCTCGGATGCTGTCATT |
| | | CAGCGCTTTTAATTTGACCT |
| | | CCAGATAGTTGCTGTATTTC |
| | | TGTTCCCATTGGCTGCTGCG |
| | | CAGCTTCGTATAACTCGAGT |
| | | TATTGTTGCGCTCTGCCTCG |
| | | GCGTACTGGCTCATGATCTG |
| | | GATCTTGTCCGTGTCGCTTT |
| | | TCTTCGAGTGTTTCTCGCAA |
| | | ACGATGTGCACGGCCTGCAG |
| | | TGTCCAATCGGAGTCGAGCT |
| | | GGCGCCGAAACTGGCGGATC |
| | | TGAGCCTCCACACTGCCCTG |
| | | TTTCTCTATCCACGGCGGAA |
| | | CCGCCTCCTGCCGTTTCAGA |
| | | ATGTTGTTCAAGTGGTACTC |
| | | TGTGCGGTCAATGAAGGCGT |
| | | TATTGCCGGTGAAATCTTTG |
| | | GGAAGCGGTTTTCCTCGGGG |
| | | AAGATTACGAAATTCCCCGC |
| | | GTCGTTGCGCTTCCTGGATC |
| | | T |
| 4 | MOX Terminator | CAATCTCCGGAATGGTGATC |
| | | TGATCGTTCCTGAAAACCTC |
| | | GACATTGGCTCCCTCCTGAC |
| | | ACAGGTACTCGTACAGGTTC |
| | | CAGGTAAACGAGTCGTAGTT |
| | | GTCGATCATGACAACGTTCT |
| | | TAGAAGCGGCCGGCATTTTG |
| | | AAGGTGACTAATAGCCTAAG |
| | | AAAATATTTAATTTAATTTT |
| | | CATTAAATTTTCCTATACTC |
| | | GCTATTTCAGCTTTTCATCT |
| | | CATCACTTCATAAACGATAT |
| | | AAACCAGAAAAAGAACTATT |
| | | TTCAAACACGCTTCTCAAAA |
| | | GCGGTATGTCCTTC+NLCAC |
| | | GTCTCCTTAGAATCTGGCAA |
| | | GTCCGCGAGGG |
| 5 | Sc URA3 | GTTTCAATTCATCATTTTTT |
| | auxotrophic | TTTTATTCTTTTTTTTGATT |
| | marker gene | TCGGTTTCCTTGAAATTTTT |
| | in the | TTGATTCGGTAATCTCCGAA |
| | expression | CAGAAGGAAGAACGAAGGAA |
| | plasmid | GGAGCACAGACTTAGATTGG |
| | | TATATATACGCATATGTAGT |
| | | GTTGAAGAAACATGAAATTG |
| | | CCCAGTATTCTTAACCCAAC |
| | | TGCACAGAACAAAAACCTGC |
| | | AGGAAACGAAGATAAATCAT |
| | | GTCGAAAGCTACATATAAGG |
| | | AACGTGCTGCTACTCATCCT |
| | | AGTCCTGTTGCTGCCAAGCT |
| | | ATTTAATATCATGCACGAAA |
| | | AGCAAACAAACTTGTGTGCT |
| | | TCATTGGATGTTCGTACCAC |
| | | CAAGGAATTACTGGAGTTAG |
| | | TTGAAGCATTAGGTCCCAAA |
| | | ATTTGTTTACTAAAAACACA |
| | | TGTGGATATCTTGACTGATT |
| | | TTTCCATGGAGGGCACAGTT |
| | | AAGCCGCTAAAGGCATTATC |
| | | CGCCAAGTACAATTTTTTAC |
| | | TCTTCGAAGACAGAAAATTT |
| | | GCTGACATTGGTAATACAGT |
| | | CAAATTGCAGTACTCTGCGG |
| | | GTGTATACAGAATAGCAGAA |
| | | TGGGCAGACATTACGAATGC |
| | | ACACGGTGTGGTGGGCCCAG |
| | | GTATTGTTAGCGGTTTGAAG |
| | | CAGGCGGCAGAAGAAGTAAC |
| | | AAAGGAACCTAGAGGCCTTT |
| | | TGATGTTAGCAGAATTGTCA |
| | | TGCAAGGGCTCCCTATCTAC |
| | | TGGAGAATATACTAAGGGTA |
| | | CTGTTGACATTGCGAAGAGC |
| | | GACAAAGATTTTGTTATCGG |
| | | CTTTATTGCTCAAAGAGACA |
| | | TGGGTGGAAGAGATGAAGGT |
| | | TACGATTGGTTGATTATGAC |
| | | ACCCGGTGTGGGTTTAGATG |
| | | ACAAGGGAGACGCATTGGGT |
| | | CAACAGTATAGAACCGTGGA |
| | | TGATGTGGTCTCTACAGGAT |
| | | CTGACATTATTATTGTTGGA |
| | | AGAGGACTATTTGCAAAGGG |
| | | AAGGGATGCTAAGGTAGAGG |
| | | GTGAACGTTACAGAAAAGCA |
| | | GGCTGGGAAGCATATTTGAG |
| | | AAGATGCGGCCAGCAAAACT |
| | | AAAAAACTGTATTATAAGTA |
| | | AATGCATGTATACTAAACTC |
| | | ACAAATTAGAGCTTCAATTT |
| | | AATTATATCAGTTATTACCC |
| | | GGGAATCTCGGTCGTAATGA |
| | | TTTTTATAATGACGAAAAAA |
| | | AAAAATTGGAAAGAAAAC |
| 6 | Myoglobin | MGLSDGEWQLVLNAWGKVEA |
| | Amino Acid | DVAGHGQEVLIRLFTGHPET |
| | sequence | LEKFDKFKHLKTEAEMKASE |
| | | DLKKHGNTVLTALGGILKKK |
| | | GHHEAEVKHLAESHANKHKI |
| | | PVKYLEFISDAIIHVLHAKH |
| | | PSDFGADAQAAMSKALELFR |
| | | NDMAAQYKVLGFHG |

1-100. (canceled)

101. An Ogataea polymorpha biomass comprising an amino acid content of 0.1-25% w/w wherein said biomass is dried.

102. The Ogataea polymorpha biomass of claim 101, further comprising 0.1-2% w/w of histidine.

103. The Ogataea polymorpha biomass of claim 101, further comprising 0.03-3.1% w/w of heme.

104. The Ogataea polymorpha biomass of claim 101, wherein said biomass is a lysed or hydrolyzed biomass.

105. A flavor precursor mixture comprising the Ogataea polymorpha biomass of claim 101.

106. The flavor precursor mixture of claim 105, further comprising at least one compound selected from the group consisting of: proteins, amino acids, carbohydrates, vitamins, and a mixture thereof.

107. The flavor precursor mixture according to claim 105, wherein the Ogataea polymorpha comprises at least 0.001% w/w of a compound selected from heme, glutathione, histidine and a mixture thereof.

108. The flavor precursor mixture according to claim 105, wherein the Ogataea polymorpha biomass is an extract or a hydrolyzed extract.

109. A food product comprising an Ogataea polymorpha biomass.

110. The food product of claim 109, wherein the Ogataea polymorpha biomass is a lysed or hydrolyzed biomass.

111. The food product of claim 109, wherein the Ogataea polymorpha biomass comprises at least 0.001% w/w of a compound selected from heme, glutathione, histidine and a mixture thereof.

112. The food product of claim 109, wherein said product is selected from the group consisting of: sauces, marinades, condiments, dressings, brines, broths, soups, tofu, tempeh, seitan, meat analogs, meat products, extended meat products, cultured meat products, and dietary supplements.

113. A dietary supplement comprising 0.01-80% w/w of the Ogataea polymorpha biomass of claim 101.

114. The dietary supplement of claim 113, wherein the biomass is a lysed or hydrolyzed biomass.

115. The dietary supplement of claim 113, wherein the Ogataea polymorpha biomass comprises at least 0.001% w/w of a compound selected from heme, glutathione, histidine and a mixture thereof.

116. A method to produce an Ogataea polymorpha biomass comprising 0.03-3.1% w/w of heme, the method comprising:
a) culturing and propagating O. polymorpha cells, in a media culture comprising a substance capable of inducing an oxidative stress to O. polymorpha cells,
b) optionally lysing the cells,
c) optionally adjusting the pH, and
d) drying and/or concentrating by removing water to obtain a solid O. polymorpha biomass;
wherein the temperature of the method is below 65° C.

117. The method of claim 116, wherein the substance capable of inducing oxidative stress is methanol.

118. The method of claim 117, wherein the biomass is lysed or hydrolyzed before drying or concentrating.

119. The method of claim 118, wherein the hydrolysis of the biomass consists of hydrolyzing the macromolecules to compounds of about 25 kDa, about 20 kDa, about 15 kDa, about 10 kDa, about 5 kDa, about 2 kDa, about 1 kDa, and/or 05 kDa.

120. The method of claim 119, further comprising a filtration step to remove particles greater than 50 kDa.


SEQUENCE LISTING (6 sequences)

SEQ ID NO: 1 - DNA, 1102 bp, Bos taurus
  source 1..1102: mol_type = other DNA
aaaaaccccagctgtcggagacaggacacccagtcagtccgcccttgttctttttctcttcttcagactgcgccatggggctcagcgactgggaatggcagttggtgctgaatgcctgggggaaggtggaggctgatgtcgcaggccatgggcaggaggtcctcatcaggctcttcacaggtcatcccgagaccctggagaaatttgacaagttcaagcacctgaagacagaggctgagatgaaggcctccgaggacctgaagaagcatggcaacacggtgctcacggccctggggggtatcctgaagaaaaagggtcaccatgaggcagaggtgaagcacctggccgagtcacatgccaacaagcacaagatccctgtcaagtacctggagttcatctcggacgccatcatccatgttctacatgccaagcatccttcagacttcggtgctgatgcccaggctgccatgagcaaggccctggaactgttccggaatgacatggctgcccagtacaaggtgctgggcttccatggctaagccccacccctgtgcccctcaccccacccacctgggcagggtgggcggggactgaatcccaagtagttatagggtttgcttctgagtgtgtgctttgtttaggagaggtgggtggaagaggtggatgggttaggggtggagggagccttgggagaggcctggggaccaggctttcagtggagggtcatcaacttgggaaccatgagaagcttgactgtggctggctgagtctgggtcaaactcaactttcctttcacctcaatgccaacccaattcctaccaacctctaaactgacctgcacctttaccctcaccttaaatccccaatccgagctgtcaacataaactccagcctaattctctgaccccatcacccagccccttgaagacagcagagtgtcttgcttgccctgagaaggaagtgtgggccgggtgggacggccacacccagccctagggaggcatggaggcatggtgtctgcaacataaatgtcccttctcaggtaggggagtgacacctggtttaataaaggatttctcacatcacaaaaaaaaaaaaaaaa

SEQ ID NO: 2 - DNA, 1158 bp, synthetic construct
  source 1..1158: mol_type = other DNA
gtttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaattggaaagaaaac

SEQ ID NO: 3 - DNA, 1066 bp, synthetic construct
  source 1..1066: mol_type = other DNA
cgatgaaggcagagagcgcaaggaggcggtatttatagtgccattcccctctctgagagacccggatggtagtcgagtgtatcggagacagcttgatgtagactccgtgcctgccggctcctcttattggcggacaccagtgagacaccccggaacttgctgtttttctgcaaaatccggggtgaccagtgggagcctatttgcacacacgagcgggacaccccactctggtgaagagtgccaaagtcattctttttcccgttgcggggcagccgattgcatgttttaggaaaatattacctttgctacaccctgtcagatttaccctccacacatatatattccgtcacctccagggactattattcgtcgttgcgccgccagcggaagatatccagaagctgttttccgagagactcggttggcgcctggtatatttgatggatgtcgcgctgcctcacgtcccggtacccaggaacgcggtgggatctcgggcccatcgaagactgtgctccagactgctcgcccagcaggtgtttcttgatcgccgcctctaaattgtccgcgcatcgccggtaacatttttccagctcggagtttgcgtttagatacagtttctgcgatgccaaaggagcctgcagattataacctcggatgctgtcattcagcgcttttaatttgacctccagatagttgctgtatttctgttcccattggctgctgcgcagcttcgtataactcgagttattgttgcgctctgcctcggcgtactggctcatgatctggatcttgtccgtgtcgcttttcttcgagtgtttctcgcaaacgatgtgcacggcctgcagtgtccaatcggagtcgagctggcgccgaaactggcggatctgagcctccacactgccctgtttctctatccacggcggaaccgcctcctgccgtttcagaatgttgttcaagtggtactctgtgcggtcaatgaaggcgttattgccggtgaaatctttgggaagcggttttcctcggggaagattacgaaattccccgcgtcgttgcgcttcctggatct

SEQ ID NO: 4 - DNA, 328 bp, synthetic construct
  source 1..328: mol_type = other DNA
caatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgaggg

SEQ ID NO: 5 - DNA, 1158 bp, synthetic construct; identical to SEQ ID NO: 2
  source 1..1158: mol_type = other DNA

SEQ ID NO: 6 - protein, 154 aa, Bos taurus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG